(19) United States
(12) Patent Application Publication
(10) Pub. No.: US 2023/0263657 A1
(43) Pub. Date: Aug. 24, 2023

(54) HEATER ASSEMBLIES FOR TEMPERATURE MANAGEMENT SLEEVES

(71) Applicant: FLOTHERM, INC., Los Angeles, CA (US)

(72) Inventors: Douglas M. PATTON, Irvine, CA (US); Bradley C. LIANG, Bloomfield Hills, MI (US); Abhinav RAMANI, Los Angeles, CA (US); Brian T. KANNARD, Los Angeles, CA (US); Peter Luke SANTA MARIA, Emerald Hills, CA (US); Morgan Taylor MCKEOWN, River Heights, UT (US)

(21) Appl. No.: 18/131,281

(22) Filed: Apr. 5, 2023

Related U.S. Application Data
(63) Continuation of application No. PCT/US21/54169, filed on Oct. 8, 2021.
(60) Provisional application No. 63/089,884, filed on Oct. 9, 2020.
(60) Provisional application No. 63/116,594, filed on Nov. 20, 2020.

(51) Int. Cl. A61F 7/02; A61H 9/00

(52) U.S. Cl. CPC A61F 7/02; A61H 9/0092; A61H 2201/5056; A61H 2201/0207; A61H 2205/12; A61F 2007/0045

(57) ABSTRACT

A temperature-management device comprises a wearable sleeve including a foot portion configured to cover at least a portion of a sole of a foot of a patient, a calf portion configured to cover at least a portion of a calf of the patient, the calf portion having one or more compression bladders associated therewith, and a popliteal fossa portion configured to cover at least a portion of a popliteal fossa of the patient. The temperature-management device further comprises a heater assembly configured to be secured to the wearable sleeve, the heater assembly including a foot heating pad, a popliteal fossa heating pad, and a physical connector extending between the foot heating pad and the popliteal fossa heating pad.

### CROSS-REFERENCE TO RELATED APPLICATION(S)

This application is a continuation of Int'l Application No. PCT/US21/54169, filed Oct. 8, 2021, entitled HEATER ASSEMBLIES FOR TEMPERATURE MANAGEMENT SLEEVES, which claims priority to the following applications: U.S. Provisional Application No. 63/089,884, filed Oct. 9, 2020, entitled TEMPERATURE MANAGEMENT SLEEVES; and U.S. Provisional Application No. 63/116,594, filed Nov. 20, 2020, entitled HEATER ASSEMBLIES FOR TEMPERATURE MANAGEMENT SLEEVES, the disclosures of which are hereby incorporated by reference in their entirety.

### BACKGROUND

### Field

The present disclosure relates to the field of medical procedures and devices.

### Description of Related Art

Each year, over 60 million surgical procedures are performed in the United States. Patient temperatures can drop precipitously during surgery due to the effects of general anesthesia, lack of insulating clothing, and/or exposure to cold operating room temperatures.

### SUMMARY

Described herein are systems, devices, and methods to facilitate patient normothermia maintenance in connection with certain medical procedures. In particular, systems, devices, and methods in accordance with one or more aspects of the present disclosure can facilitate the heating of patient core body temperature during a medical procedure through the use of certain wearable heating and/or compression systems, which may advantageously reduce the risk of contracting hypothermia and/or other complications associated with low core body temperature over certain periods of time.

In some implementations, the present disclosure relates to a temperature-management device comprising a wearable sleeve including a foot portion configured to cover at least a portion of a sole of a foot of a patient, a calf portion configured to cover at least a portion of a calf of the patient, the calf portion having one or more compression bladders associated therewith, and a popliteal fossa portion configured to cover at least a portion of a popliteal fossa of the patient. The temperature-management device further comprises a heater assembly configured to be removably secured to the wearable sleeve, the heater assembly including a foot heating pad, a popliteal fossa heating pad, and a physical connector extending between the foot heating pad and the popliteal fossa heating pad.

The foot portion can include a heel pad, such as an inflatable heel pad configured to be fluidly coupled to at least one of the one or more compression bladders. For example, such an inflatable heel pad can be fluidly coupled to a channel having a one-way valve associated therewith. In some embodiments, the popliteal fossa portion of the wearable sleeve has a knee cut-out, such that the wearable sleeve is open around a knee of the patient. The physical connector of the heater assembly may include electrical wiring contained within a connector portion of a fluid-tight pouch.

In some embodiments, the calf portion of the wearable sleeve is detachable from one or more of the foot portion or the popliteal fossa portion. The foot heating pad can have a visual indicator that corresponds with a corresponding indicator associated with the foot portion of the wearable sleeve. The wearable sleeve can include a closable cover configured to be covered over one or more portions of the heater assembly when the heater assembly is secured to the wearable sleeve. In some embodiments, the temperature-management device further comprises an extension component configured to connect to the calf portion over a shin of the patient. In some embodiments, the temperature-management device further comprises a fluid interface that is heat-sealed to the wearable sleeve and includes three fluidly-isolated conduits in fluid communication with three compression bladders of the one or more compression bladders, respectively.

In some embodiments, the heater assembly includes one or more color-coded indicators indicating a size of the heater assembly and the wearable sleeve includes one or more corresponding color-coded indicators on indicating a size of the wearable sleeve relative to the heater assembly. The wearable sleeve can include an adhesive strip configured to secure the heater assembly to the wearable sleeve. The wearable sleeve can include a heater assembly delineation pad on which the heater assembly is configured to be secured.

In some embodiments, the wearable sleeve and the heater assembly are configured such that, when the heater assembly is secured to the wearable sleeve, the foot heating pad and the popliteal fossa heating pad are disposed on an inside of the wearable sleeve and at least a portion of the physical connector is disposed on an outside of the wearable sleeve. For example, the at least a portion of the physical connector can be configured to run outside of the calf portion of the wearable sleeve when the heater assembly is secured to the wearable sleeve. In some embodiments, the heater assembly is configured such that the physical connector runs along a front or side of a leg of the patient between the foot and popliteal fossa when the heater assembly is secured to the wearable sleeve.

In some implementations, the present disclosure relates to a heater assembly comprising a first heating pad, a second heating pad, electrical wiring electrically coupling the first heating pad and the second heating pad to an electrical connector, and a pouch including a first heating pad portion covering at least a portion of the first heating pad, a second heating pad portion covering at least a portion of the second heating pad, and an elongate connector portion extending between the first heating pad portion and the second heating pad portion, the elongate connector portion covering at least a portion of the electrical wiring.

The electrical connector can project substantially perpendicularly from the connector portion. The heater assembly may further comprise a means for indicating a usage of the heater assembly. For example, the means for indicating can comprise a mechanical indicator. As another example, the means for indicating can comprise one or more light sources. The heater assembly may comprise control circuitry configured to determine a number of uses of the heater assembly and activate an indicator in response to said determination.

In some embodiments, the heater assembly further comprises one or more temperature sensors on a patient-facing side of one or more of the first heating pad and the second heating pad. The heater assembly may further comprise thermally-conductive gel disposed on at least a portion of a patient-facing side of one or more of the first heating pad and the second heating pad. In some embodiments, the first heating pad and the second heating pad comprise resistive heating devices.

The connector portion may follow a straight path between the first heating pad portion and the second heating pad portion. In some embodiments, the connector portion has one or more bends configured to allow for the connector portion to traverse a non-linear path.

In some implementations, the present disclosure relates to a method of treating a patient. The method comprises providing a wearable sleeve including one or more compression bladders, providing a heater assembly including a first heating pad, a second heating pad, and a connector portion that physically coupled between the first heating pad and the second heating pad, securing the heater assembly to the wearable sleeve, securing the wearable sleeve on a limb of a patient, compressing the limb of the patient using the one or more compression bladders, applying heat to a sole of a foot of the limb using the first heating pad, and applying heat to a popliteal fossa of the limb using the second heating pad. The method can further comprise inflating a heel pad of the wearable sleeve. In some implementations, compressing the limb, applying heat to the sole of the foot, and apply heat to the popliteal fossa are performed simultaneously

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an embodiment of a surgical system including a patient-heating subsystem in accordance with one or more embodiments.

FIG. 2 illustrates an embodiment of a surgical system including a patient-heating subsystem in accordance with one or more embodiments.

FIG. 3 is a block diagram including components of a temperature-management system in accordance with one or more embodiments.

FIG. 4 illustrates a side view of a temperature-management sleeve system secured to a patient in accordance with one or more embodiments.

FIG. 5 illustrates components of a temperature-management system in accordance with one or more embodiments.

FIGS. 6-1, 6-2 illustrate a heater assembly in configurations with and without a pouch encasement, respectively, in accordance with one or more embodiments.

FIG. 7 is an exploded view of an example heater assembly in accordance with one or more embodiments.

FIG. 8 is an exploded view of an example heater assembly in accordance with one or more embodiments.

FIG. 9-1 shows a temperature-management sleeve, including a detailed cross-sectional view of one or more heater portions, in accordance with one or more embodiments.

FIGS. 9-2A, 9-2B, and 9-2C show heating pad layers of a heating assembly in accordance with one or more embodiments.

FIG. 9-2D shows a cross-sectional view of a heating pad of a heater assembly in accordance with one or more embodiments.

FIG. 10 shows a heater assembly including a mechanical usage indicator in accordance with one or more embodiments.

FIG. 11 shows a heater assembly including an electrical usage indicator in accordance with one or more embodiments.

FIG. 12 illustrates a side view of a temperature-management system having a heater assembly secured thereto in accordance with one or more embodiments.

FIG. 13-1A illustrates a temperature-management system having a heater assembly contained therein in accordance with one or more embodiments.

FIG. 13-2A illustrates a temperature-management system having a heater assembly including a pouch container associated therewith in accordance with one or more embodiments.

FIGS. 13B-13D show views of a fluid connector in accordance with one or more embodiments.

FIG. 14 shows a temperature-management sleeve having a heater assembly disposed and/or contained between layers of a sleeve article in accordance with one or more embodiments.

FIG. 15-1 shows a temperature-management sleeve having a heater assembly cover in accordance with one or more embodiments.

FIG. 15-2 shows the temperature-management sleeve of FIG. 14 with the heater assembly cover in a closed configuration in accordance with one or more embodiments.

FIG. 16A shows a temperature-management sleeve having an adhesive coupling feature and a heater assembly in accordance with one or more embodiments.

FIG. 16B shows the temperature-management sleeve of FIG. 16A with the heater assembly secured to the adhesive coupling feature in accordance with one or more embodiments.

FIGS. 17A and 17B show a temperature-management sleeve having a heater assembly associated therewith with heater covers in open and closed configurations, respectively, in accordance with one or more embodiments.

FIG. 18 shows a temperature-management sleeve having certain dimensions in accordance with one or more embodiments.

FIGS. 19-21 show color-coordinated temperature-management sleeves and associated heater assemblies in accordance with one or more embodiments.

FIG. 22 is a flow diagram illustrating a process for managing patient temperature in accordance with one or more embodiments.

FIG. 23A shows a temperature-management sleeve extension component in accordance with one or more embodiments.

FIGS. 23B and 23C shows a temperature-management sleeve disposed on a lower limb of a patient with the sleeve extension component of FIG. 19A in detached and attached configurations, respectively, in accordance with one or more embodiments.

FIGS. 24A-24C illustrate a wearable sleeve and heater assembly in various configurations and/or coupling states in accordance with one or more embodiments.

FIG. 25 shows a temperature-management sleeve disposed on a limb of a patient in accordance with one or more embodiments.

FIG. 26 shows a cut-away view of a heater assembly in accordance with one or more embodiments.

FIGS. 27-1, 27-2, 27-3, 27-4, and 27-5 show stages of a manufacturing process for manufacturing a heater assembly in accordance with one or more embodiments.

FIGS. 27-6A and 27-6B show top and bottom perspective views, respectively, of a heater assembly in accordance with one or more embodiments.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer.” “inner,” “upper.” “lower,” “below,” “above,” “vertical.” “horizontal,” “top.” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

### Overview

The present disclosure relates to systems, devices, and methods for maintaining normothermia for a patient, such as during a medical procedure/surgery. Each year, over 60 million surgical procedures are performed in the United States. While great care may be taken to prevent surgical complications, one commonly overlooked and under-addressed problem is the risk of developing hypothermia before, during, or after surgery (referred to as “inadvertent perioperative hypothermia” or “IPH”). For example, patient temperatures can drop precipitously during surgery due to the effects of general anesthesia, lack of insulating clothing, and/or exposure to cold operating room temperatures. Even with modern standards of care, approximately 30-50% of surgical patients may develop hypothermia.

Hypothermia often causes much more than patient discomfort. Patients who suffer even mild IPH can face a significantly elevated risk of developing surgical site infections, cardiac morbidities, intraoperative bleeding, and other avoidable complications. Together, these complications can significantly increase recovery time and overall length of hospital stay, leading to increased costs for all parties. By some estimates, the unmanaged risk for IPH is a $15 billion problem in the United States alone, and yet it is largely overlooked.

Perioperative heat loss can occur predominantly via convective heat transfer, particularly through the palms of the hands, soles of the feet, and exposed surgical site surface area. During preoperative care, patients are often dressed solely in a gown and are often exposed to relatively cold waiting areas with little to no insulation. Although patients are generally only anesthetized at the start of surgery, patients often arrive at the surgical theater moderately hypothermic. This can put a patient at greater risk for developing severe hypothermia once anesthesia has been administered. Postoperative drops in core temperature can increase the likelihood of developing additional comorbidities, such as morbid cardiac outcomes, surgical site infections, and blood loss, any of which can prolong recovery and hospitalization.

Patients undergoing surgery can develop hypothermia during the surgical procedure itself, especially when the procedure involves the patient's core area, such as procedures involving the posterior or anterior sides of the thoracic, abdominal, and pelvic regions. Surgeries of the core involve the exposure of vital internal organs to the colder environment and thus carry a greater risk of hypothermia. Furthermore, core surgeries often necessitate uncovering of the trunk and chest, which render blankets and many other currently-available interventions inadequate. Once in the operating room, patients may be naked and exposed to a room temperature well below 36 degrees Celsius and to cold liquids used to wash the surgical site during sterilization preparation. At the onset of surgery, delivered anesthetics can immediately impair the normal autonomic thermoregulatory controls. Colder blood may be transferred from the peripheries of the body to the core through a phenomenon known as redistributive hypothermia. Vasodilatation and reduction in muscle tone can cause a significant drop in core temperature within the first half-hour of surgery.

Overall, compared to non-hypothermic patients, those who suffer from IPH experience greater rates of surgical site infections, bleeding, and cardiac complications. Such issues may require additional monitoring and/or increase the length of stay and/or subjective discomfort. The development of IPH is strongly correlated with a multitude of physiological organ system changes impacting the cardiovascular, respiratory, neurologic, immunologic, hematologic, drug-metabolic, and wound-healing mechanisms. The incidence of several post-surgical complications can be increased due to even mild hypothermia.

Intraoperatively, hypothermia can cause a decrease in cardiac output and heart rate, which can lead to ventricular dysrhythmias. Platelet functions can become impaired and there can be a decrease in coagulation factors, which can, in turn, lead to greater intraoperative bleeding and blood loss. Impaired immune functions can increase the rate of surgical site infections. Hypothermia is associated with a four-fold increase in surgical wound infection and twice as many morbid cardiac events. In select procedures such as colorectal, gynecologic, or spinal surgery, where infection rates are normally higher than other surgeries, hypothermia can be exceedingly dangerous to the intraoperative and postoperative recovery. These complications and others are supported in multiple studies and can result in both clinical and economic burdens.

Current methods of preventing hypothermia often are not completely effective. Even with the current interventions, up to 46% of patients are reported to be hypothermic at the start of surgery, and 33% are hypothermic upon arrival to the post-anesthesia care unit (PACU). Assuming the cost savings for maintaining normothermia in one patient is approximately $5.000 per patient, and approximately 30% of the 17 million high-risk surgical patients are hypothermic, a system-wide cost savings of $15 billion could be realized by keeping these patients normothermic. With rising healthcare costs and recent initiatives mandating the maintenance of perioperative normothermia, hospital administrators are in need of new, efficacious and cost-effective devices to address perioperative hypothermia, a product space that has seen little innovation since the introduction of the forced-air warming blanket nearly three decades ago.

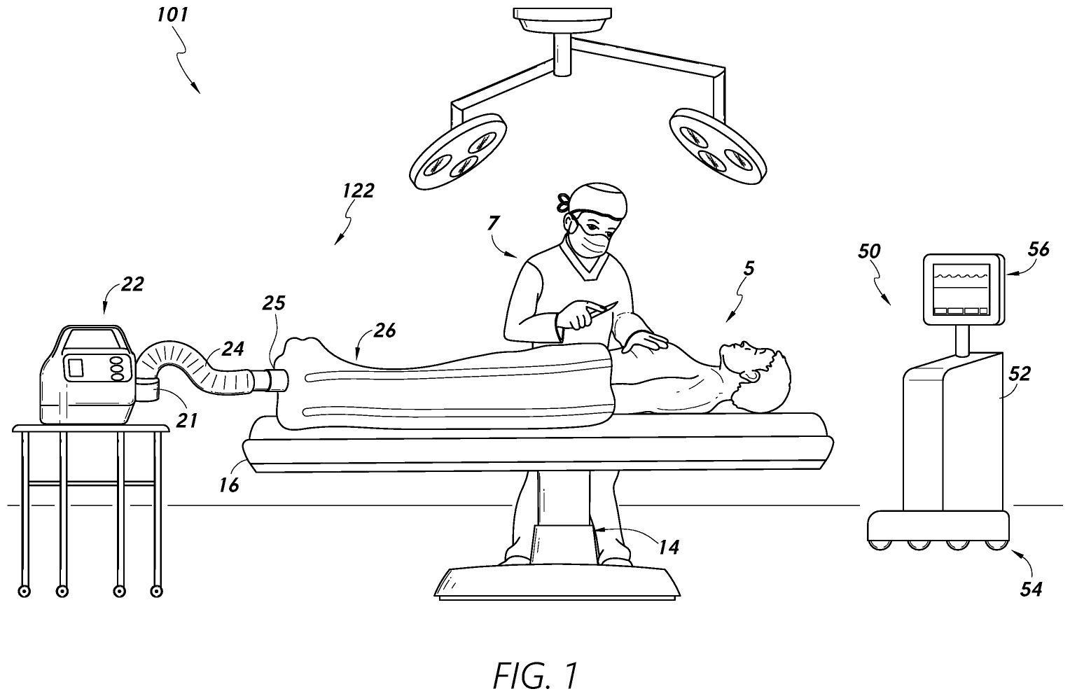

Some solutions for perioperative warming may implement forced-air temperature management devices (e.g., warming blankets). Some temperature management solutions utilize high-heat transfer conduction heating blankets and intraoperative hand-warming devices. For example, FIG. 1 illustrates an embodiment of a surgical system 101 including a forced-air, blanket-type patient-heating subsystem 122 in accordance with one or more embodiments. The system 101 may be implemented within a surgical environment, such as an operating room or other patient treatment environment or facility. The patient-heating subsystem 122 may be implemented as a means of preventing the core body temperature of the patient 5 from dropping below certain levels and/or dropping below desired levels for certain periods of time, which may result in hypothermia and/or other medical complications or adverse health effects for the patient 5. The operating environment may include a table 16 supported by a base 14, as well as one or more monitor systems 51, which may include one or more displays 56, towers 52, and/or mobilization features 54.

The patient-heating subsystem 122 may include a blanket-type structure or device 26, which may be draped and/or covered over at least a portion of the patient's body. For example, in connection with some procedures, the surgical operation(s) may require access to the chest or core of the patient. In such procedures, the blanket device 26 may be placed over at least a portion of the lower extremities and/or core of the patient 5. That is, the blanket device 26 may generally be placed over an area of the patient's body that is not presently being operated on to avoid interference with the surgical procedure/access. In some implementations, the blanket device 26 may have certain openings, apertures, slits, cutouts, or the like formed therein to provide access to the relevant parts of the patient anatomy, while still allowing for the blanket device 26 to be disposed generally about/near the access portion(s).

In some embodiments, the blanket device 26 may be configured to direct thermal energy to portions of the patient anatomy covered thereby. For example, in some implementations, the blanket device 26 may operate by having heated fluid, such as air, circulated and/or filled within one or more portions of the blanket device 26, such as through one or more channels thereof. For example, warm air may be circulated through the blanket device 26, wherein such air may be introduced into the blanket device 26 via one or more ports 25, wherein fluid may flow in a fluid circuit and/or exit the blanket device 26 via one or more ports, apertures, and/or other egress channels/pathways. The heated air/fluid can be provided by a fluid source unit 22, which may comprise one or more air compressors or the like and/or one or more heating elements for heating air provided thereby. The heated fluid (e.g., air) may be provided from the fluid source unit 22 to the port 25 via a hose 24 and/or hose interface 21.

Circulating fluid may be circulated through the blanket device 26 through one or more ports back to the fluid source unit 22 in some embodiments. Additionally or alternatively, heated air may exit the blanket device 26 via one or more patient-facing apertures or ports, which may serve to further heat the patient anatomy. In embodiments in which heated fluid exits the blanket device 26 towards the patient and/or into the surgical environment, such air may introduce certain contaminants into the surgical environment, which can result in infection or other medical issues. That is, it may be desirable for the surgical environment to be substantially sterile to prevent exposure of the patient anatomy, which may be of a particular concern with respect to internal patient anatomy that may be exposed to the surgical environment during the relevant procedure/operation. Forced-air, blanket-type heating systems may further present a relatively cumbersome physical structure that can get in the way, such as when the blanket is disposed in an area near the surgical site. Furthermore, in some situations, operating room staff may turn down the temperature on a forced-air device in consideration of their own comfort, as forced-air systems can heat the surrounding air/environment. Moreover, certain devices may not be used in preoperative warming for one or more of the following reasons, among others: (1) some devices may immobilize the upper limbs, impeding patient mobilization; (2) devices may be cumbersome (e.g., a device may float on the patient and get blown off or fall off during use and/or transport, and they require large, predominantly floor-based blowers that may not be mobile; (3) they may not attach to the patient and/or can become dislodged during transport and obstruct the bed and other monitors and devices; and (4) they can require a conscious administrative decision to implement.

Embodiments of the present disclosure advantageously provide certain improved devices, methods, and systems for maintaining a patient's core body temperature before, during, and/or after surgery. Furthermore, embodiments described herein provide methods and systems for core body temperature management in an unobtrusive, effective, and easy-to-use (e.g., easy to set-up) manner. Some embodiments of the present disclosure can be suitable for use before, during, and/or after a surgical procedure and can be acceptable to the patient while awake in the preoperative and/or postoperative settings.

In some embodiments, lower limbs of patients may be leveraged to provide therapy and/or enable mobility. For example, some devices described herein may provide flexibility and/or one or more spaces around a knee, ankle, and/or other portions of a patient's body to allow the patient to flex and/or extend the limbs. Such patient mobility may provide a variety of benefits, including allowing patients to stand up to use the restroom without removing and reapplying the device.

Some embodiments of temperature management devices disclosed herein may be configured to provide warming to one or more arteries and/or veins passing along the patient's lower limbs. Moreover, some embodiments may involve compression of one or more portions of the patient's body. For example, compression may be applied to the patient's calf. In some embodiments, compression may be performed in a sequential and/or gradient manner. Devices and systems disclosed herein advantageously combine separate sleeve structures/garments, which may include compression features, with heater assemblies that include one or more heating elements, wherein the heater assembly and the sleeve can be physically coupled to produce a combined wearable system. By utilizing separable and couplable heater assemblies and sleeve structures, such components of the temperature-management system can be used, combined, disposed of, powered, connected, and/or otherwise managed separately from one another, which can provide certain benefits with respect to patient matching, manufacturing, storage, organization, and/or other considerations.

### Temperature-Management Sleeves and Systems

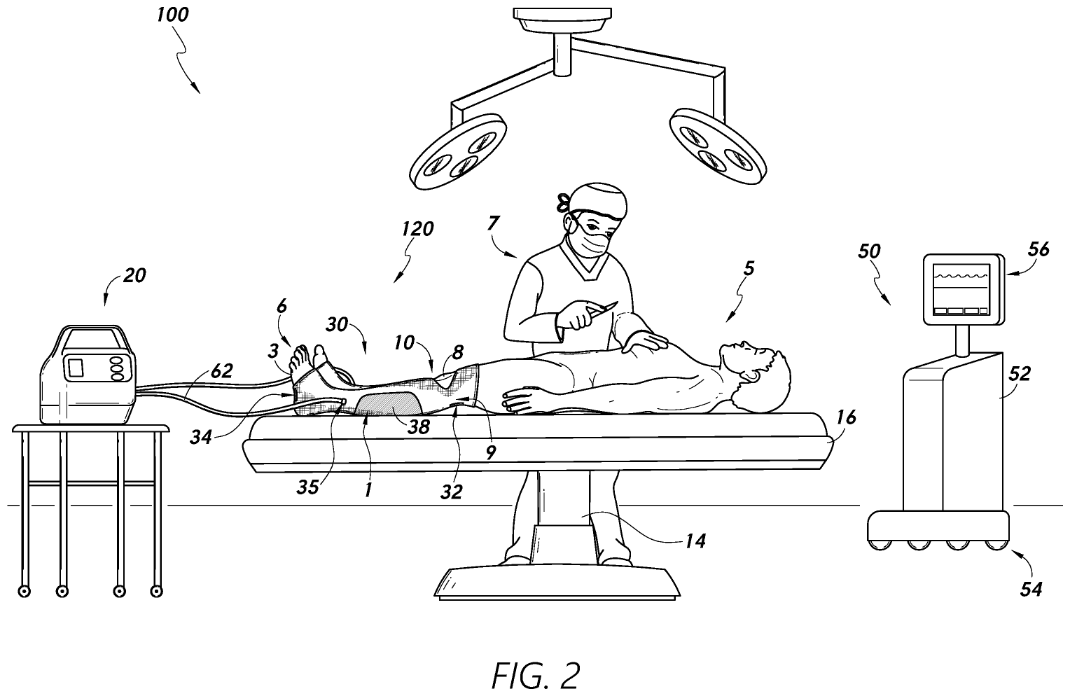

FIG. 2 illustrates an embodiment of a surgical system 100 including a patient-heating subsystem 120 in accordance with one or more embodiments. The system 100 includes a sleeve-type patient-heating subsystem 120, which can represent an alternative to a blanket-type heating system as described above with respect to FIG. 1. The sleeve-type heating subsystem 120 can include one or more temperature-management sleeves 30 disposed over and/or on portion(s) of one or more lower limbs 10 (e.g., legs) of the patient 5. The sleeve(s) 30 can include one or more heating elements/devices (referred to herein as “heaters,” “heater pads,” “heating pads,” etc.), wherein at least one of such heating elements or devices 34 is positioned on or over the sole 3 of the foot 6 of the patient 5 when the sleeve 30 is disposed on the lower limb 10, whereas another heating element/device 32 is placed on or over the popliteal fossa 9 of the lower limb 10, on an opposite side of the limb 10 from the knee 8.

The sleeve 30 may advantageously be open around the knee 8, as shown in FIG. 2, thereby providing flexibility, comfort, and/or access to the knee 8 when the sleeve 30 is disposed on the patient's leg 10. Generally, implementation of the sleeve 30 such that the knee portion is open and/or uncovered may be tenable due to the heating in that region of the leg 10 being focused on and/or limited to the popliteal fossa area 9, which is generally behind the knee on the backside of the leg 10. That is, the sleeve 30 may advantageously not heat the front of the leg in the area of the knee 8, which may be desirable to avoid overheating of the leg and/or knee portion thereof, which may result in injury and/or other complications to the patient 5. Furthermore, the absence of heating pads/devices in the area of the front of the knee can allow for a cut-out in the area of the knee, as shown and described throughout the present disclosure, which can provide improved flexibility/mobility for the limb 10 when the sleeve 30 is secured (e.g., relatively tightly) on the limb 10. Focusing and/or limiting the heating in the upper portion of the sleeve 30 to the popliteal fossa region/area 9 can be effective due to the proximity of certain blood vessels to the skin in the area of the popliteal fossa relative to other areas of the leg, such as the front side of the leg and/or knee 8, where blood vessels may not be as close to the skin, and therefore heating (or cooling) in such areas may not be as effective with respect to increasing (or reducing) and/or maintaining the core temperature of the patient 5.

The temperature-management sleeve 30 may further include one or more fluid-fillable bladders/chambers 38, which may be used for implementing intermittent compression with respect to the leg 10 when the sleeve 30 is disposed on the leg 10 of the patient 5. For example, in some implementations, intermittent compression in the calf area 1 of the leg 10 may be implemented simultaneously with the application of thermal energy/heat to the sole 3 of the foot and the popliteal fossa 9 to facilitate heating of the venous and arterial blood of the patient 5, which may serve to increase the core temperature of the patient. Although fluid-filled bladders are described herein, it should be understood that references to fluid-filled bladders or the like may refer to any type of compression element, whether inflatable, fluid-containing, or any other type of compression element. For example, such compression elements may include certain elastic compression features. In some embodiments, compression elements associated with sleeve devices/structures of the present disclosure can include physical straps and/or smart material(s) configured to implement squeezing and/or relaxing around at least a portion of the calf. In some embodiments, circulation stimulation of the limb 10 may be implemented using electrical stimulation provided by one or more electrodes associated with the sleeve device 30, wherein such stimulation may be in the calf area 1 of the sleeve.

The patient-heating subsystem 120 can include a control unit, device, or system 20, which may be equipped with one or more fluid-compression devices, electrical power sources, and/or certain control circuitry configured to control the operation of the heating pads/devices 32, 34 and/or compression bladder(s) 38. In some embodiments, the sleeve device 30 includes one or more temperature sensors, which may be generally associated with and/or positioned near the heating pads/devices 32, 34, wherein signals generated by such sensors may be provided to the control unit 20 to provide feedback that may direct operation of the control unit with respect to the application of heating and/or compression to the leg 10. The control unit 20 may include one or more fluid and/or electrical tubes, cables, wires, and/or other connectors 62, which may be used to provide fluid (e.g., air) to the bladder(s) 38 and/or electrically drive the heaters 32, 34 to provide heat to the patient 5. In some embodiments, the control unit 20 is embodied at least in part in/on the sleeve assembly 30.

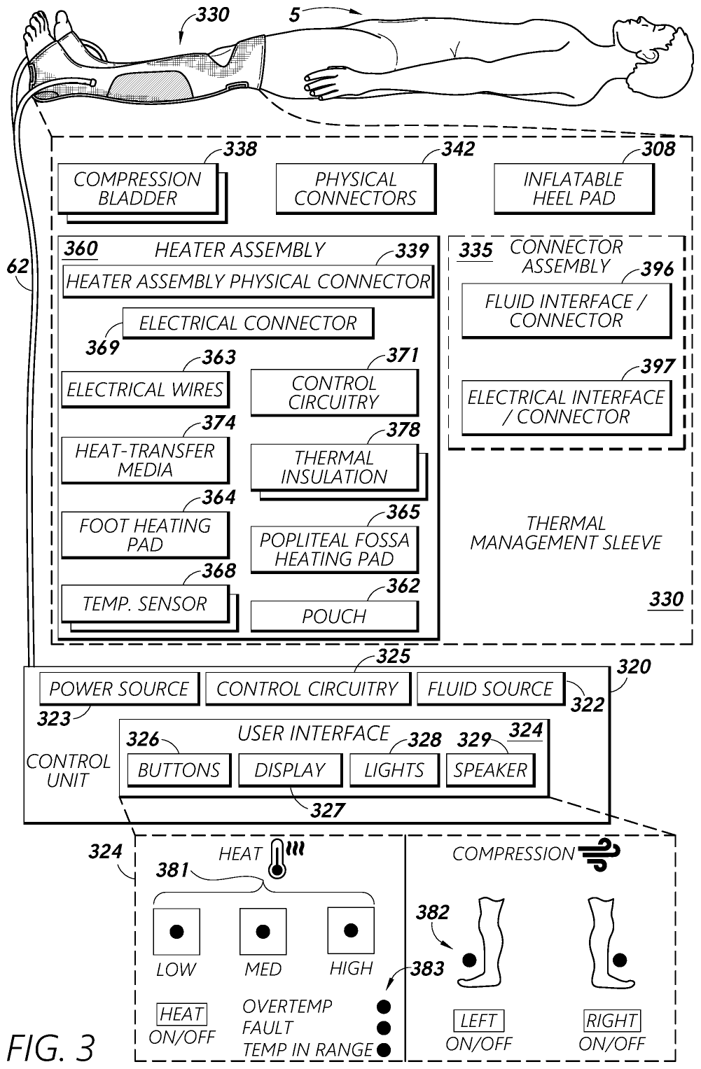

FIG. 3 is a block diagram including example components of a temperature-management system in accordance with one or more embodiments of the present disclosure. The system includes a temperature-management sleeve 330, which may comprise a body-wearable structure/article/garment configured to be disposed about at least a portion of a limb of the patient 5, such as a lower limb/leg. The term “sleeve” is used herein according to its broad and ordinary meaning, and may refer to any type of sleeve, garment, article, brace, cuff, sock, boot, wrap, or the like configured to be placed and/or secured on or about a limb or other body part of a person. The thermal-management sleeve 330 can include one or more physical connectors 342, which may include one or more straps, clips, clasps, folds, elastically-deformable materials or components, wraps, or other physical components of the sleeve that may be used to secure the sleeve to the limb of the patient.

The sleeve 330 can include one or more compression bladders 338, which may be configured to receive fluid through one or more fluid interfaces/connectors 396 from a fluid source 322 of a control unit or system 320. In some embodiments, the sleeve 330 includes 2, 3, or more compression bladders, which may be individually inflatable and/or depletable to provide sequential compression of the limb of the patient. Although described as compression bladders for convenience, it should be understood that the feature(s) 338 may comprise any type of compression elements. In some embodiments, certain electrical wiring or other electrical connectivity component(s) may be coupled to each compression element 338 for providing electrical power to the respective compression element. For example, electrical current may be provided to the compression element(s) 338 to initiate mechanical tightening or relaxing of the element(s) in order to provide sequential compression of the limb of the patient.

In some embodiments, the sleeve 330 includes an inflatable heel pad 308, which may be fluidly coupled to one or more fluid channels associated with the compression bladder(s) 338. For example, the inflatable heel pad 308 may be configured to receive fluid/air through a one-way fluid valve from one or more of the compression bladder(s) 338 and/or fluid channels associated therewith. The inflatable heel pad 308 may advantageously reduce the risk and/or effects of pressure ulcers (e.g., bedsores) and/or other friction- or pressure-based physical injury that may occur to the heel of the limb on which the sleeve 330 is disposed due to, for example, prolonged positioning of the heel and/or repeated/agitated movement of the heel on the bed or other surface, which may occur during the course of a surgical operation. Although described in connection with certain embodiments as being an inflatable heel pad, it should be understood that any reference herein to a heel pad may be inflatable or non-inflatable. For example, the heel pad 308 can comprise foam or other cushioned material and/or form, which may be associated with a heel portion of the sleeve structure. The heel pad 308 may be a donut-/torus-shaped balloon or foam form.

The sleeve 330 may further have associated therewith a heater assembly 360, which may be removably attached or secured to the sleeve 330 using one or more aspects of the physical connector(s) 342 and/or other attachment means or mechanisms of the sleeve 330. The heater assembly 360 can include a plurality of heating pads/devices disposed within a pouch-type container 362. For example, the pouch 362 may comprise one or more layers of plastic that envelop at least a portion of the heating pads/devices 364, 365. In some embodiments, the heater assembly 360 is contained between fabric layers of the sleeve 330. For example, the heating assembly may be stitched or welded in place in/on the sleeve 330. In such embodiments, the heating assembly may not be contained in a pouch-type container 362 (e.g., polymer pouch), but rather the heating pads and connecting wires may be stitched/welded into/onto the sleeve 330.

The heating pads of the heater assembly 360 may include a foot heating pad 364 and a popliteal fossa heating pad 365. The term “heating pad” is used herein according to its broad and ordinary meaning, and may refer to a heating element that has a relatively broad surface configured to apply heat over a broad (e.g., relatively flat and/or contoured) area, such as an area of the sole of the foot and/or area of the popliteal fossa, or any other area corresponding to the form factor of the heating element/pad. Furthermore, the term “heating pad,” as used herein, may refer to the heating element itself, such as a resistive heating element implemented on an at least partially flat substrate or in an at least partially flat plane or form, and/or may be used to refer to a portion of a heater assembly including a heating element and one or more layers or components associated therewith and/or disposed adjacent thereto.

Although embodiments are described herein in the context of heating, it should be understood the various embodiments of temperature management systems and devices disclosed herein can be implemented to provide cooling to the limb of a patient. That is, any reference herein to heating, heating pads, heater assemblies, and/or other heating systems, processes, devices, or the like can be understood to refer to cooling, cooling pads, cooling assemblies, and/or other cooling systems, processes, devices, or the like. Furthermore, it should be understood that the various devices, systems, and processes disclosed herein can be used/implemented to perform or incorporate both heating and cooling functionality. In some embodiments, cooling using a cooling pad is implemented utilizing thermoelectric cooling element(s).

The structure of sleeves disclosed herein may include certain holes or other attachment features for attaching various components of a temperature-management system, including heater/cooler assemblies, physical connectors, tubes, pads, or other features. For example, certain straps (e.g., velcro) may be implemented to attach various components.

Certain electrical wiring and/or other type(s) of conductive tracing(s) 363 may generally be coupled to each of the foot heating pad 364 and the popliteal fossa heating pad 365 for providing electrical power to the respective heating elements/devices. For example, electrical current may be provided through the electrical wires 363 and through resistive conductor(s) of the respective heating pads to generate radiative heat. That is, the foot heating pad 364 and the popliteal fossa heating pad 365 may operate as resistive heaters that are configured to heat certain portions of the patient's anatomy through infrared/heat radiation. It should be understood that any references herein to electrical wiring can be understood to refer to any type of electrically-conductive tracing, or any other signal-transmission medium, means, or mechanism. For example, such signal-transmission means may comprise a pneumatic signal transmission system, which may include an air/gas-filled tube used for signal transmission in some implementations.

In some embodiments, the heating pads 364, 365 are associated with certain thermal insulation features 378, which may be disposed on a side of the heating pads that is generally away from the patient when the sleeve 330 is disposed on the patient's limb. Such insulation may reduce the risk of burn or damage from contact with the outside of the sleeve. Such insulation may further serve to direct heat in the direction of the patient rather than outwardly away from the patient when the heating pads are activated. In some embodiments, heat-transfer media 374 may be applied to one or more heating surfaces or areas of the respective heating pads 364, 365, wherein such transfer media may advantageously promote thermal conduction/transfer from the heating pads to the patient's skin. In some embodiments, heat transfer media can be applied to a patient-facing surface/area of the heating pad(s) 364, 365 and/or sleeve 330 to facilitate heat transfer between the sleeve and the patient's skin.

In some embodiments, the heater assembly 360 includes certain control circuitry 371, which may be implemented on one or more circuit boards or other electrical modules/devices electrically coupled to the wiring 363 in some manner. For example, in some embodiments, the control circuitry 371 may be implemented at least in part in one or more boards and/or chips disposed adjacent to and/or on one or more of the heating pads 364, 365. For example, the heating pads may comprise substrates on/in which resistive heating conductors are disposed, wherein the control circuitry is implemented in/on an area of the substrate that is not covered by the resistive heating conductors. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The heater assembly 360 includes physical connector component(s) 339 that physically connect between the foot heating pad 364 and the popliteal fossa heating pad 365 to allow for the heater assembly 360 to span the distance between the popliteal fossa and the sole of the foot of the patient in a manner such that the heater assembly 360 provides a substantially unitary structure that can be placed and span the distance between the popliteal fossa and the sole of the foot. For example, the physical connector 339 may include one or more portions of the electrical wires 363 that connect between the heating pads, and/or between a respective heating pad and an electrical connector 369 of the heater assembly that is configured to be coupled to an electrical interface/connector 397 of the thermal-management sleeve 330. For example, the electrical connector 369 may be configured to be plugged into the electrical interface/connector 397 of the sleeve 330. In some embodiments, the electrical connector 369 is configured to plug directly into one or more cables 62 coupled to the control unit 320. For example, the connector 369 may be configured to be exposed externally from the sleeve 330 when the heater assembly is positioned in/on the sleeve 330.

The heater assembly 360 may further comprise one or more temperature sensors 368, such as one or more thermistors or the like. The temperature sensor(s) 368 may be configured to generate signals indicative of temperatures present and/or experienced at one or more portions of the heater assembly 360 and/or thermal management sleeve 330. For example, the temperature sensors 368 may provide signals indicating temperatures associated with the respective heating pads and/or areas of the sleeve and/or patient anatomy that are disposed/situated in proximity thereto. The temperature sensor(s) 368 may be configured to provide sensor signals to the electrical connector 369 and/or to the control unit 320 via the electrical connector 369 and/or electrical interface/connector 397. The temperature sensor(s) 368 may be disposed on a patient-facing side of the foot 364 and/or popliteal fossa 365 heating pads.

The connector assembly 335 of the thermal-management sleeve 330 may include one or more connectors for coupling to a fluid source 322 of/from the control unit 320, a power source 323 of/from the control unit 320, and/or the electrical connector 369 of the heater assembly. Although the connector assembly 335 is illustrated as including one or more separate fluid interface/connector 396 and electrical interface/connector 397 modules/components, it should be understood that such connectivity features may be implemented in a single connector structure in some embodiments. Furthermore, in some embodiments, either or both of the fluid interface/connector 396 and the electrical interface/connector 397 may be implemented in two or more separate connectors. For example, the electrical interface/connector 397 may comprise a first electrical connector for receiving electrical power from the control unit 320 as well as a second electrical connector for electrically coupling to the electrical connector 369 of the heater assembly 360 to provide power thereto and/or to receive certain electrical signals (e.g., temperature sensor signals) therefrom.

The temperature-management system shown in FIG. 3 further includes a control unit 320. It should be understood that although a single box is used to illustrate the control unit 320, the functionality and/or components associated therewith may be implemented in separate devices, systems, modules, and/or components. The control unit 320 may include a power source 323 configured to provide power to the thermal-management sleeve for powering, for example, the heating elements, the control circuitry 371, and/or temperature sensor(s) 368 of the heater assembly 360. The control unit 320 may further include a fluid source 322, which may comprise one or more air compressors or the like. The fluid source(s) 322 may be used to provide fluid/air to the compression bladder(s) 338 for implementation of sequential compression in the catheter area of the sleeve 330.

The control unit 320 include certain control circuitry 325 configured to implement any of the various functional operations disclosed herein in connection with temperature management solutions of the present disclosure. For example, the control circuitry 225 may be configured to determine when and/or to what degree to activate the compression bladder(s) 338, foot heating pad 364, popliteal fossa heating pad 365, temperature sensor(s) 368, and/or user input/output functionality. The control unit 320 may further comprise certain user interface components, which may further allow for user engagement with the control unit 320 through provision of user input and/or notification/indication of certain information relating to the operation of the control unit 320. For example, the user interface component(s) 324 may include one or more user input buttons and/or one or more user output displays 327, lights 328, speakers or other audible notification means or mechanisms 329, or other input/output components for communication with a user. Example user interface representations are shown in block 324, which shows certain lights or other visual indicators 381 indicating a heating level of one or more heating pads of the heater assembly 360, and/or certain output indicators 383 indicating other conditions of the heater assembly. Additional indicators 382 may be provided to indicate which limb is presently subject to intermittent compression and/or the degree to which such compression is implemented.

In some embodiments, the sleeve 330 includes a pump attachment, such as a mini-pump, which may serve as a fluid source for intermittent compression and/or heated or cooled fluid circulation in the sleeve. The pump attachment may be secured to the calf portion of the sleeve in some implementations. The various components of the thermal management sleeve 330 can be configured to be battery powered. For example, the sleeve may be configured to include one or more battery packs or the like, which may be electrically coupled to the heater assembly 260 and/or one or more components thereof. The battery power source can be a component of the heater assembly in some embodiments. For example, the battery source can be contained within the pouch 362, which may advantageously provide protection from fluids and/or other contaminants for the batter(ies).

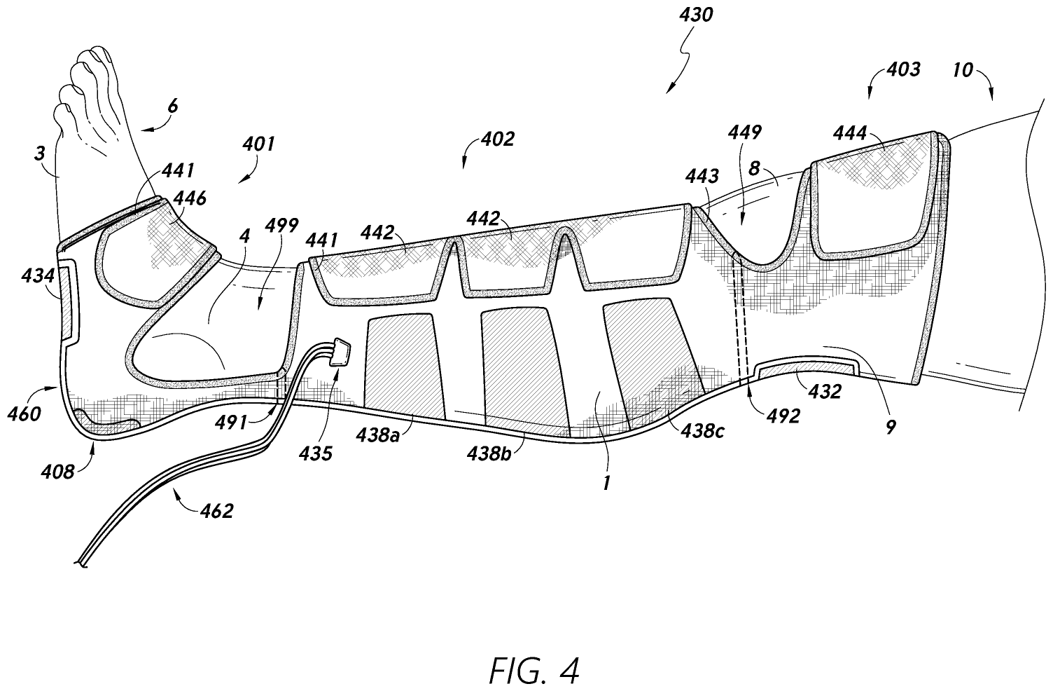

FIG. 4 illustrates a side view of a temperature-management sleeve system 430 secured to a limb 10 of a patient in accordance with one or more embodiments of the present disclosure. The temperature-management sleeve 430 includes a plurality of heating pads, including a first heating pad 434 disposed in a region of the sleeve 430 that is configured to be placed on and/or in proximity to the sole 3 of a foot 6 of the patient. The sleeve 430 further includes a second heating pad/device 432 that is configured to be situated/disposed on or adjacent to the popliteal fossa region 9 of the patient's limb 10, which may generally be on a backside of the limb 10 opposite the knee 8. The heating pads 432, 434 may be part of a heater assembly 460 that is attachable and/or configured to be coupled to the sleeve 430.

The sleeve 430 further includes a calf portion 402, which may be associated with one or more compression bladders 438, which may be implemented to provide sequential compression for the promotion of blood flow within the limb 10 and/or prevention/treatment of deep vein thrombosis. In some embodiments, sequential compression may be implemented in the calf portion 402 simultaneously with heating of the sole of the foot 3 and the popliteal fossa 9 using the heating pads 434 and 432, respectively. Such simultaneous implementation/execution of the heating and sequential compression as provided by the sleeve system 430 can advantageously provide improved heating and/or temperature maintenance for a patient during a surgical procedure, thereby reducing the risk of hypothermia development as described in detail herein.

In some embodiments, the calf portion 402 of the sleeve 430 is detachable from a foot portion 401 and/or a popliteal fossa portion 403 of the sleeve 430. For example, the sleeve 430 may include a detachable feature or region 491 configured to allow for detachment of the foot portion 401 from the calf portion 402. For example, such detachment/attachment means 491 may comprise a Velcro coupling, and/or other type of clip, strap, tie, snap, or other physical attachment means, mechanism or tearaway feature, wherein the structure of the foot portion 401 can be separated from and/or attached to the calf portion 402. The detachability of the foot portion 401 and/or popliteal fossa portion 403 from the calf portion 402 may allow for the calf portion 402 and associated sequential compression functionality to remain on the limb 10 and/or be implemented even after removal of the foot 401 and/or popliteal fossa 403 portions of the sleeve 430, which may be desirable for the purpose of an increasing patient comfort and/or allowing for patient mobility in certain situations.

The sleeve 430 includes certain physical connectors/couplings, such as one or more calf portion straps 442, foot portion straps 446, and/or popliteal fossa portion straps 444, which may serve to secure the sleeve 430 to the limb 10. The sleeve 430 may further include certain flexibility-enhancement features, such as a knee cut-out/opening 449 and/or ankle cut-out/opening 499. The ankle opening 499 can advantageously allow for rotation of the limb 10 at the ankle 4 with reduced discomfort and/or physical strain on the sleeve 430. The knee opening 449 may advantageously allow for bending of the limb 10 at the knee 8 with reduced obstruction and/or discomfort to the patient.

The sleeve 430 includes a connector assembly 435, which may comprise a physical connector for a fluid source, which may provide heated or non-heated fluid for inflation of the compression bladder(s) 438 and/or heel pad 408, and/or may allow for electrical coupling of the heater assembly 460 associated with the heating pads 432, 434 with an electrical power source, which may be provided from a control unit or the like (not shown in FIG. 4; see FIGS. 2, 3). In some embodiments, the sleeve 430 includes certain visual indicator/color-coordination features 441, 443, which may be used to associate the sleeve 430 with an appropriate heater assembly that is configured and/or designed to fit and/or be attached to the sleeve 430. For example, the heater assembly may have corresponding color indicators indicating the match/association between the heater assembly and the sleeve 430. The color-coordination features 441, 443 may be associated with outer trim, band(s), or similar structural features of the sleeve 430.

### Heater Assemblies and Temperature-Management Sleeves

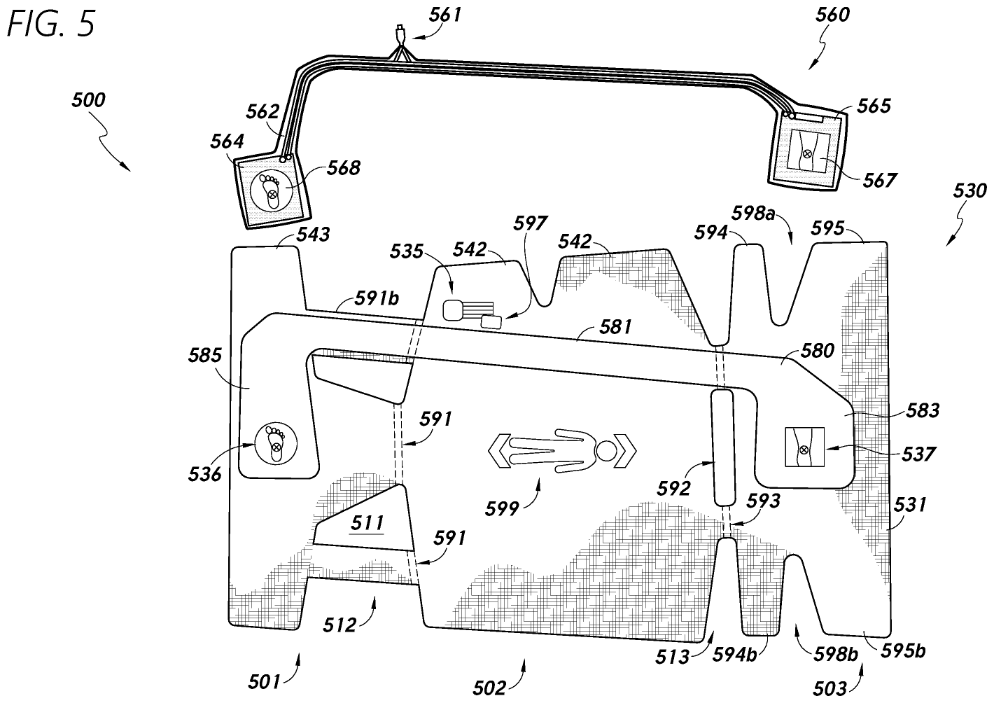

FIG. 5 illustrates components of a temperature-management system 500 including a sleeve 530 and a heater assembly 560 in accordance with one or more embodiments of the present disclosure. The sleeve 530 includes a foot portion 501, calf portion 502, and a popliteal fossa portion 503. In some embodiments, the various portions of the sleeve 503 may be detachable from one another. In particular, as shown, the device 530 may include certain attachment/detachment features 591 configured to allow for detachment and/or attachment of the foot portion 501 to the calf portion 502. Such attachment/detachment feature(s) may comprise any attachment or detachment means disclosed herein in connection with any of the disclosed embodiments. Additionally or alternatively, the sleeve 530 may comprise certain attachment/detachment features 593 configured to allow for detachment and/or attachment of the popliteal fossa portion 503 from the calf portion 502, as shown. Such attachment/detachment features 593 may comprise any attachment or detachment means disclosed herein.

The sleeve 530 further comprises certain straps or couplings 542, 543, 594, 595, which may be used to strap or secure the sleeve 530 to a limb of a patient. Certain visual indicator(s) 599 indicating a direction of the sleeve relative to the limb of the patient and/or orientation of the patient may further be included on one or more sides of the sleeve 530.

Sleeve 530 may further include a seating area or feature 585 on which a heater assembly 560 may be placed, disposed or otherwise secured to the sleeve 530. Features having characteristics that are similar to the feature 585 may be referred to herein as heater assembly delineation features, components, or portions. For example, such features may generally outline the footprint/profile of the heater assembly and provide support, protection, and/or visual direction/indication therefore. Although illustrated with heater assembly delineation features in some instances, it should be understood that the various embodiments of sleeves disclosed herein may or may not include such features.

In some embodiments, the delineation feature 585 may include certain visual indicators 536, 537 indicating positions of placement for a foot heating pad 564 and popliteal fossa heating pad 565, respectively, to assist a user in identifying the correct orientation of the heater assembly 560 relative to the sleeve 530. For example, a first indicator, marking, or other feature 536 may indicate that a foot heating pad is to be placed in the area associated therewith, wherein a corresponding indicator 568 may be associated with the heater assembly 560, wherein the markers/indicators 536, 568 indicate a match and relative placement of the heater pad 564 of the heater assembly 560 relative to the delineation area/feature 585 of the sleeve. Likewise, the popliteal was a heating pad 565 may have associated therewith a visual indicator 567 indicating the placement thereof in the area identified by the marking 537 on the sleeve 530. The respective markings 568, 567 and/or 536, 537 may include visual images indicating the parts of the patient anatomy associated therewith, namely the sole of the foot and the popliteal fossa, as shown in FIG. 5. The sleeve 530 may further include a connector 535 configured to receive a fluid source connector for receiving air for compression chambers or the like, as well as an electrical connector 597 for plugging-in a corresponding electrical connector 561 of the heater assembly 560 into the sleeve 530. The sleeve 530 may in turn receive electrical power from an electric connection/cable provided from a control unit or other power source.

The sleeve 530 may advantageously include certain gaps 598 for providing an opening around the patient's knee when the sleeve 530 is disposed on the leg of the patient to thereby allow for flexibility around the knee as well as to prevent the sleeve 530 from being placed with the heating pad 565 positioned over the knee as opposed to the popliteal fossa, which would represent a substantially less effective heating position for the heating pad 565 compared to the popliteal fossa area where the relevant blood vessels are closer to the skin. The illustrated side of the sleeve shown in FIG. 5 may be a patient-facing side of the sleeve, or may alternatively be a side of the sleeve that faces generally away from the patient, wherein the opposite side of the sleeve 530 may be placed against the limb of the patient. The sleeve 530 can further include an ankle opening 511 and/or ankle gap 512, which may advantageously provide improved flexibility and/or comfort in the area around the ankle of the patient. The sleeve 530 can further include a gap 513 below the popliteal fossa area of the sleeve to provide flexibility and/or comfort in the area between the popliteal fossa and calf of the patient.

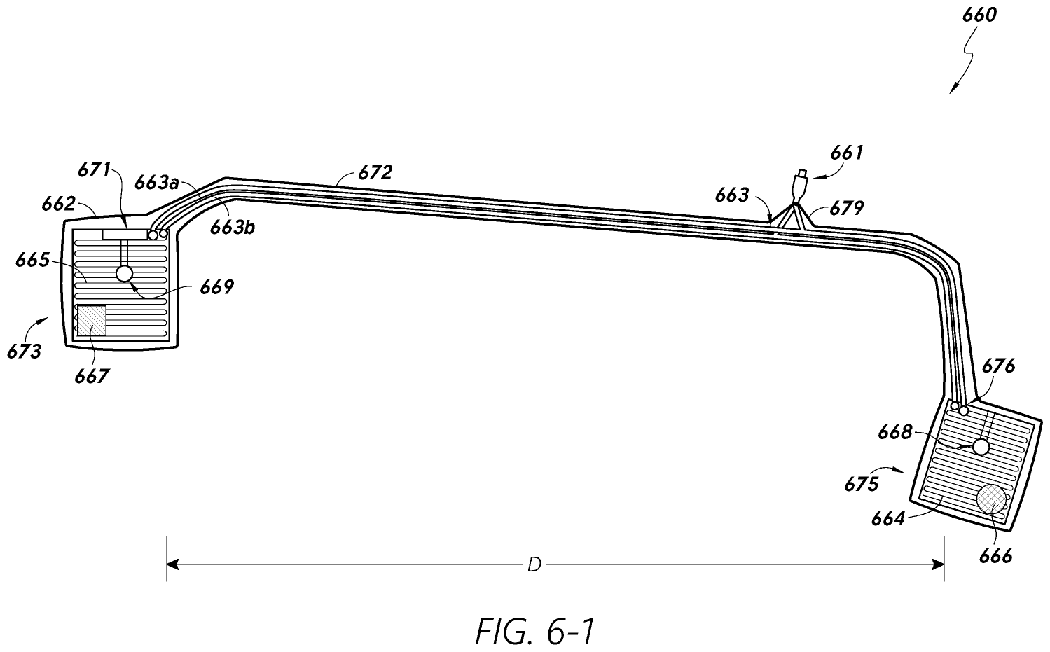

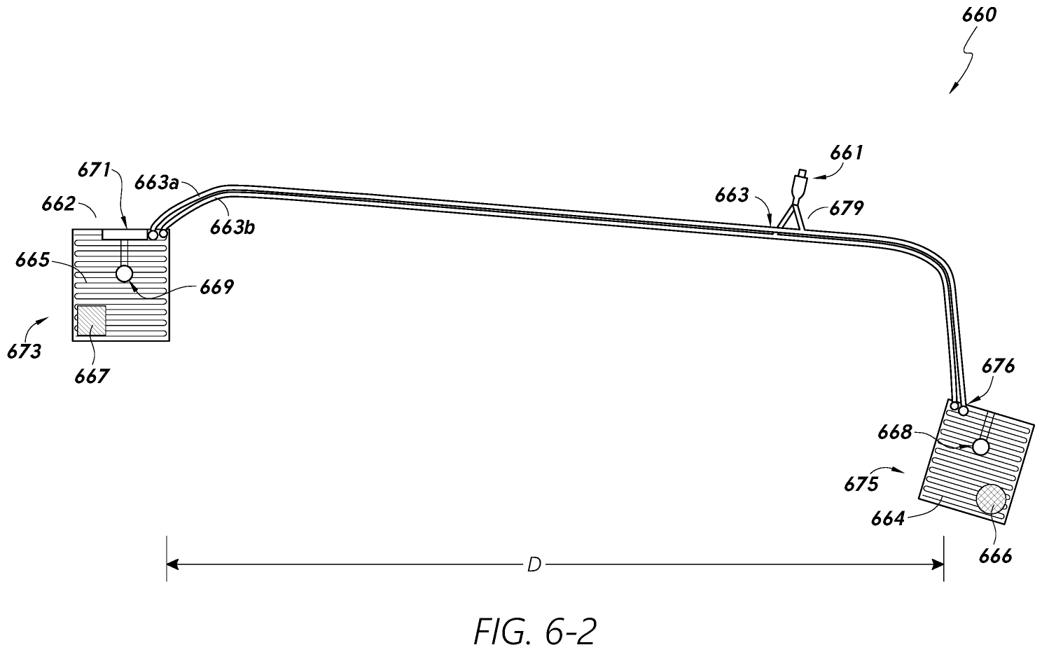

FIG. 6-1 illustrates a heater assembly 660 in accordance with one or more embodiments of the present disclosure. The heater assembly 660 can be secured to or otherwise placed/disposed on or secured or attached to a wearable sleeve structure, such as a compression-enabled sleeve device as described herein. The heater assembly 660 may advantageously span a distance D, which represents the distance between a sole of a foot of the patient and the popliteal fossa area of the patient, such that the heater assembly 660 can be positioned with a first heating pad 665 thereof positioned at or near the sole of the foot, while a second heating pad 664 is positioned at or near the popliteal fossa of the patient when the heater assembly 660 is coupled to the wearable sleeve. In some embodiments, at least some of the components of the heater assembly 660 are contained within a pouch, bag, pack, or other similar type container or structure 662. The term “pouch” is used herein according to the broad and ordinary meaning and may refer to any lining, covering, bag, container, compartment, pocket, envelope, or the like, whether sealed or at least partially open and/or whether flexible or rigid in whole or in part. With respect to the heater assembly 660, the pouch 662 may advantageously provide a fluid seal to prevent various electronics and/or other components disposed therein from contamination and/or damage.

FIG. 6-2 shows the heater assembly 660 without a pouch 662 enveloping the wires, heating pads, and/or other component(s) of the heater assembly 660. For example, the heater assembly 660 shown in FIG. 6-2 can be stitched, welded, or otherwise secured to a sleeve member/article without a pouch. In such implementations, the heater assembly 660 may be stitched, welded, or otherwise secured between layers of fabric or other material of the sleeve member/article, at least with respect to one or more portions of the heater assembly. The heater assembly 660 of FIG. 6-2 may be manufactured as a unit and stitched/welded in/to a sleeve as a unit. When contained within layers of the sleeve, the connector 660 may be passed through an aperture in a sleeve layer (e.g., outer layer facing away from the patient) for connection with an electrical connector.

With further reference to FIGS. 6-1 and/or 6-2, the heater assembly 660 includes a connector portion 672, which spans the distance D between the first heating pad 665 and the second heating pad 664. In some embodiments, electrical wiring 663 configured to provide electrical power to the respective heating pads and/or receive temperature sensor signals and/or other data signals from the thermistors 669, 668 and/or control circuitry 671 is coupled to the respective heating pads. With respect to the embodiment of FIG. 6-1, the wiring 663 may be routed/contained within a connector portion 672 of the pouch 672 to an outlet portion 679 of the pouch 662, which may be positioned/disposed along the connector portion 672 of the heater assembly 660 and/or the pouch 662. In some embodiments, the connector portion 672 may comprise an elongate sealed portion of the pouch 662.

The popliteal fossa heating pad 665 and the foot heating pad 664 may include certain resistive electrical conductors configured to have electrical current passed therethrough, wherein such current produces radiant heat (e.g., in the form of infrared radiation), which may be used to provide heating to the patient. In some embodiments, with respect to the embodiment of FIG. 6-1, the pouch 662 includes a popliteal fossa heating portion 673 configured to enclose the popliteal fossa heating pad 665. In some embodiments, the pouch portion 673 may have associated therewith one or more visual indicators or markings 667 indicating the association of the pouch portion 673 and heating pad 665 with the popliteal fossa and/or popliteal fossa portion/region of the sleeve to which the heater assembly 660 is configured to be coupled. Such marker/indicator 667 may comprise an image of the relevant physical anatomy, one or more shapes or icons indicating the anatomy, and/or certain color coordination features indicating the position (e.g., color indicator(s)/marker(s) corresponding to similar color indicator(s)/marker(s) found on the relevant portion of the sleeve to which the heater assembly 660 is to be coupled). The position indicator/marker 667 may be disposed on the pouch portion 673, or alternatively may be disposed on our coupled to the heater pad 665.

The heating pad 665 may have associated therewith one or more thermistors 669. For example, the thermistor(s) 669 may be disposed on or in proximity to a patient-facing side of the heating pad 665, which side is shown in the illustrations of FIGS. 6-1 and 6-2. In some embodiments, the markings/indicators 667, 666 are associated with a side of the heater assembly 660 that faces away from the patient when secured to the sleeve (i.e., the side of the heater assembly not visible in the image of FIG. 6). The thermistor(s) 669 may be used to provide sensor signals indicating a temperature of the heating pad 665 and/or proximate area, which may include the skin/tissue of the patient in the area of the popliteal fossa and/or the temperature of the sleeve proximate to the heating pad 665.

In some embodiments, the heater assembly 660 may include control circuitry 671, which may be configured to perform certain functionality or the heater assembly 660. For example, the control circuitry 671 may be configured to receive and/or provide electrical signals to/from the thermistor(s) 669/668. In some embodiments, the control circuitry 671 is primarily utilized for heater assembly usage-counting/tracking functionality, wherein additional control functionality is primarily handled by a control system/unit separate from the heater assembly 660. In some embodiments, the control circuitry 671 is configured to digitize thermistor readings. In some embodiments, the control circuitry 671 may be configured to activate the heating pad 665 and/or heating pad 664. That is, the control circuitry 671 may control the heating functionality of the pad(s). The control circuitry 671 may advantageously be electrically coupled to one or more of the electrical wires 663a. 663b. The control circuitry 671 may be embodied in one or more circuit boards, chips, application-specific integrated circuits (ASIC), and/or active and/or passive circuit devices (e.g., resistors, capacitors, inductors, diodes, etc.). In some embodiments, the control circuitry 671 is configured to condition certain electrical signals transferred to or from the control circuitry, such as certain filtering, amplification, multiplexing, switching, or the like. Although the control circuitry 671 is illustrated as being coupled to and/or associated with the popliteal fossa heating pad 665, it should be understood that the control circuitry 671 may be associated with the foot heating pad 664, or any region of the connector portion 672 of the heater assembly 660. The connector portion 672 may be considered to comprise the wires 663 and/or the connector portion of the pouch 662. For example, with respect to the embodiment of the FIG. 6-2, the connector portion of the heater assembly 660 can comprise only the wires connected between the heating pads and to the connector 661.

With reference to FIG. 6-1, the pouch 662 may further include a foot pouch portion 675 configured to enclose/contain the foot heating pad 664. For example, as with the popliteal fossa pouch portion 673, the foot heater pouch portion 675 may represent an expanded portion of the pouch relative to the connector portion 672 having a width and length dimension sufficient to cover the heating pad 664. The pouch portion 675 and/or heating pad 664 may include a marker/indicator 666 configured to indicate the position of the heating pad 664 and pouch portion 675 relative to a sleeve or patient. For example, such indicator/marker 666 may indicate visually the anatomy of the foot and/or the sole of the foot, or may include certain tags, color features, shapes, and/or the like indicating an anatomical position and/or correlation with a portion of the sleeve to which the heater assembly 660 is to be coupled. As with the popliteal fossa heating pad 655, the foot heating pad 664 may have associated therewith one or more thermistors 668, which may be disposed and/or associated with a patient-facing side of the heating pad 664, which side is shown in FIGS. 6-1, 6-2.

The heater assembly 660 may include an electrical connector 661, which may be electrically coupled to one or more of the wires 663 that connect to the respective heating pads 665, 664. With respect to FIG. 6-1, the electrical connector 661 may be associated with an outlet portion 679 of the pouch 662, which may have an access opening/aperture through which electrical conductor(s) may pass and exit the pouch 662. In some embodiments, a fluid seal (e.g., hermetic seal) is formed between the pouch outlet portion 679 and the electrical connector 661 and/or wires 663, such that the interior of the pouch 662 remains sealed-off from the external environment. For example, such seal may provide a sanitary barrier between the internal components of the pouch 662 and the external environment, which may advantageously prevent contamination from components contained within the pouch 662 with an external surgical field.

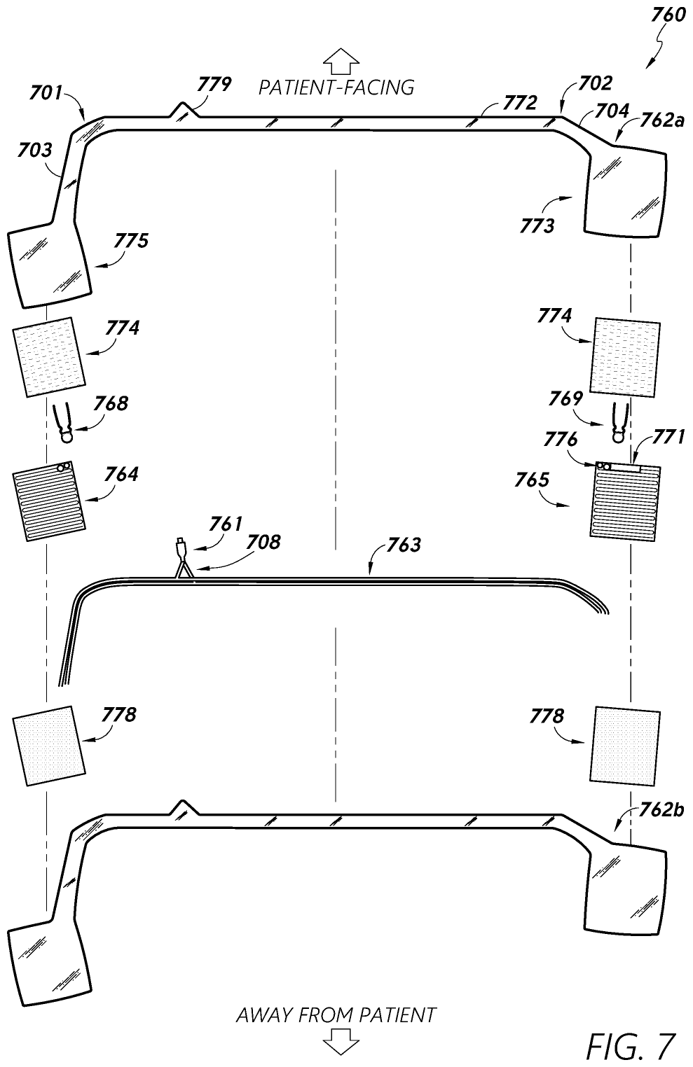

FIG. 7 is an exploded view of an example heater assembly 760 in accordance with one or more embodiments. The heater assembly 760 can include a pouch 762, which may comprise plastic, fabric, or other material, and may be configured to contain the internal components of the heater assembly 760. As described in detail herein, although shown with a pouch 762, it should be understood that the heater assembly 760, as well as any other heating assembly disclosed herein, can be manufactured without a pouch. For example, the heater assembly 760 may be configured to be sutured, welded, or otherwise secured in a sleeve member/article without a polymer or other material pouch around the heating pads and/or wiring components.

With respect to pouch-contained embodiments, the pouch 762 can include multiple segments, such as a patient-facing sheet or segment 762a as well as a segment or sheet 760b disposed on an opposite side of the heater assembly 760, such that the first segment 762a and second segment 762b are bound together, such as through heat sealing or other method, to contain/sandwich the internal components of the heater assembly 760. The segments of the pouch 762 can include various portions/regions, including a foot heater portion 775, a popliteal fossa heater portion 773, and a connector portion 772 that spans between the foot heater portion 775 and popliteal fossa heater portion 773. The connector portion 772 may further include certain areas/portions, including one or more bends 701, 702, which may allow for the connector portion 772 to navigate a path between the heating pads over/under portions of a sleeve with which heater assembly 760 is configured to be coupled. Bends in the connector portion can allow for the connector 772 to traverse a non-linear path along one or more portions of a wearable sleeve.

The heater assembly 760 further comprises certain heat-transfer media 774, which may not be present with respect to certain embodiments of heater assemblies in accordance with the present disclosure. For example, the heat-transfer media 774 may comprise heat-transfer gel and/or other media configured to facilitate the transfer/conduction of thermal energy from a respective heating pad 764, 765 through the patient-facing side of the heater assembly 760. In some embodiments, the heat transfer media 774 is disposed on, over, and/or under one or more thermistors 768, 769, which may be used to determine temperature conditions at or near the respective heating pad portions of the heater assembly 760. The thermistors 768, 769 may be disposed in proximity to the patient-facing sides of the foot 764 and popliteal fossa 765 heating pads, respectively. The control circuitry 771 may be embodied at least in part in a flexible printed circuit board having a microcontroller or other circuitry associated therewith.

In some embodiments, the heater assembly 760 includes certain control circuitry 771, which may be associated with any of the illustrated components. For example, although the control circuitry 771 is shown as disposed on or proximate to the popliteal fossa heating pad 765, it should be understood that the control circuitry 771 may be disposed on or coupled to any of the illustrated components. Furthermore, although the control circuitry 771 is illustrated as a single block, it should be understood that control circuitry associated with the heater assembly 760 may be embodied in any number of components or devices, which may be combined in an integrated unit or device, or may be physically and/or electrically separate.

The heating pads 764, 765 may be any type of heating pads, such as resistive radiating heating pads. For example, the heating pads may comprise polyimide or other substrate material, which may be flexible or rigid in whole or in part. The heating pads 764, 765, thermistors 768, 769, and/or control circuitry 771 may be coupled to electrical wiring 763, which may be electrically coupled to an electrical connector 761. In some embodiments, the electrical connector 761 is a water-resistant electrical connector. The electrical connector 761 can be coupled to the wiring 763 via a portion of the wiring 708 that juts outward from the connector portion 772 and/or routing path of the wires 763, as shown.

In some embodiments, the heater assembly 760 further includes certain thermal insulation features 778, which may comprise heat-insulating foam and/or padding. Such thermal insulation features may improve patient and/or physician comfort and/or reduce the risk of injury or damage to equipment from thermal energy originating in the heating pad 764, 765. The insulation features 778 can further advantageously present pressure on the heating pads 764, 765 to push the heating pads towards the patient to thereby improve contact and heat transfer to the patient.

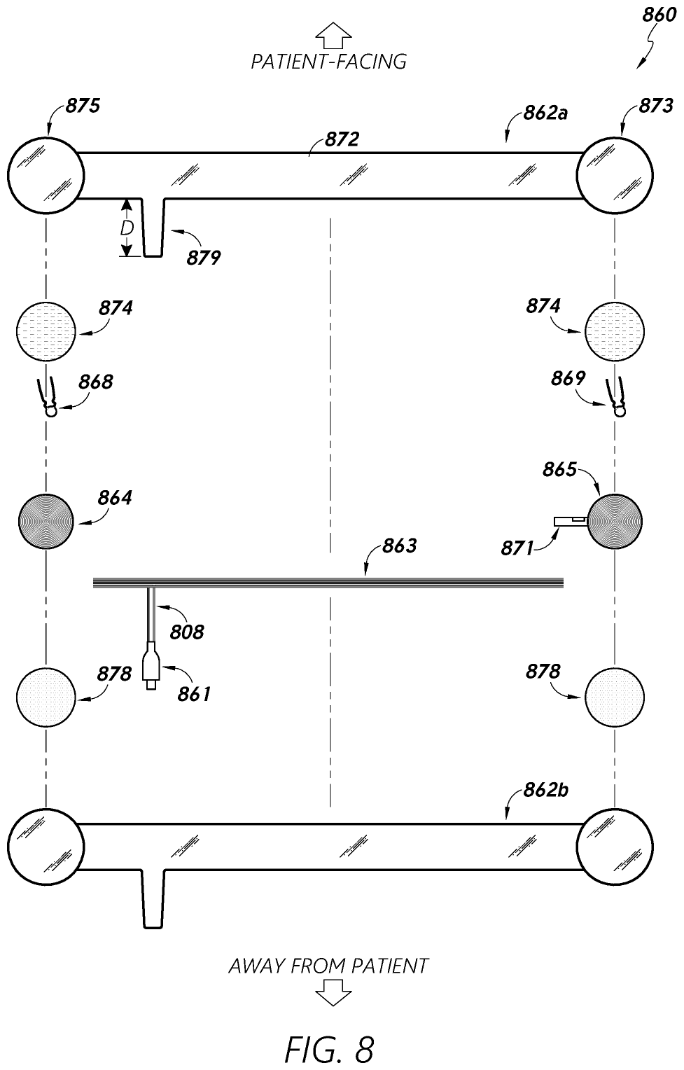

FIG. 8 is an exploded view of an example heater assembly 860 in accordance with one or more embodiments. The heater assembly 860 may be similar to the heater assembly 760 shown in FIG. 7. That is, any of the illustrated components of FIG. 8 should be understood to potentially have similar features, characteristics, and/or functionality of any counterpart/similar components illustrated in FIG. 7 and described above.

The heater assembly 860 includes a container pouch 862, which may be similar to heater assembly pouches described in connection with other embodiments herein. As described in detail herein, although shown with a pouch 862, it should be understood that the heater assembly 860 can be manufactured without a pouch. For pouch-contained embodiments, the pouch 862 may comprise a patient-facing 862a segment/portion and an away-facing 862b segment/portion. The heater pouch 862 may include a first heating pad portion 875 and a second heating pad portion 873 separated by a connector portion 872. Although some embodiments of heater assembly pouches disclosed herein include connector portions having one or more bends therein along the path between the heating pad portions, some embodiments, as illustrated in FIG. 8, include connector portions 872 that are substantially straight. That is, the connector portion 872 may traverse a generally straight-line path between the heater pad portions 875, 873.

Another apparent distinction between the various components of the heater assembly 860 compared to the heater assembly 760 illustrated in FIG. 7 relates to the generally circular shape of certain components of the assembly 860 shown in FIG. 8, whereas counterpart components shown in FIG. 7 are shown as generally rectangular (e.g., square) in shape. It should be understood that any embodiment disclosed herein may have components having any shape, as suitable or desirable for the particular application. For example, any heating pads, insulating features, thermally conductive features, heater pad pouch portions, or the like may have any type of oblong, oval, circular, rectangular, square, triangular, or any other shape or configuration.

The pouch 862 may include an electrical connector portion 879, which may jut out orthogonally from the path of the connector 872, as shown in FIG. 8. Although the portion 879 is shown as projecting substantially perpendicularly/orthogonally from the path of the connector portion 872, it should be understood that the electrical connector extension portion 879 may be formed/positioned at any acute or obtuse angle with respect to the elongate dimensions/axis of the connector portion 872 of the pouch 862. The connector portion 879 of the pouch 862 may have a length dimension D that is sufficient to extend from the position of the connector portion 872 to an electrical and/or fluid connector associated with a sleeve to which the heater assembly 860 is configured to be coupled. For example, in some implementations, when attached to the sleeve (not shown in FIG. 8), the connector portion 872 may lie in an area of the sleeve that is generally associated with a back of the calf of the patient, whereas the relevant electrical and/or fluid connector(s) to which the connector 861 connects may be removed/offset from the back of the calf by a certain distance. For example, the connector of the sleeve or other connector to which the electrical connector 861 of the assembly 860 is configured to be connected may be positioned on a side of the leg or generally towards a side or front of the leg of the patient. Therefore, the pouch extension 879 may necessarily or desirably extend a sufficient distance to provide desired fluid sealing for the pouch 862 around the electrical wires 808 connected to the connector 861.

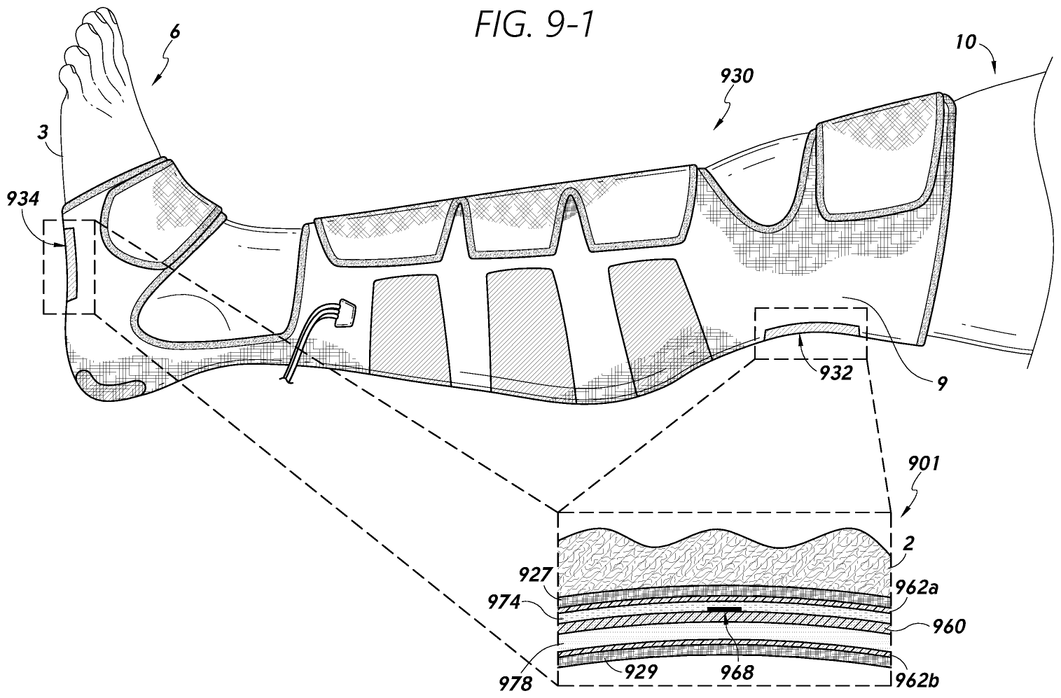

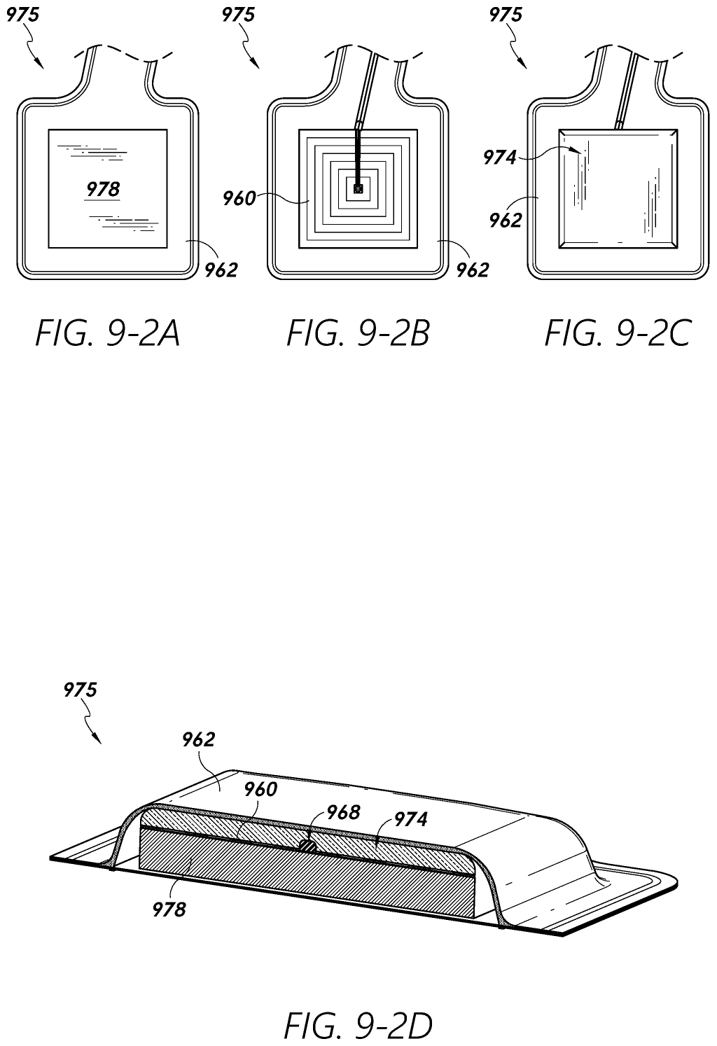

FIG. 9-1 shows a temperature-management sleeve 930, including a detailed cross-sectional view of one or more heating portions thereof, in accordance with one or more embodiments of the present disclosure. The temperature-management sleeve 930 includes a foot heating pad or portion 934 and a popliteal fossa heating pad or portion 932. For example, the foot heating portion 934 may be positioned on or proximate to the sole 3 of a foot 6 of the patient, whereas the popliteal fossa heating portion 932 may be positioned on or proximate to the popliteal fossa 9 of the patient's leg 10.

The respective heating portions of the sleeve 930 may be embodied at least in part in heater portions of a heater assembly as described in detail in connection with various embodiments disclosed herein. For example, such heater assemblies may include foot and popliteal fossa heating portions, wherein heating pads or other heating elements are disposed within a pouch, as shown in FIGS. 7 and 8 and described in detail above. The detailed image 901 represents a cross-sectional view of an example stack of a heater portion of a heater assembly and relevant/associated portions of the sleeve 930 positioned or disposed around or adjacent to the heating pad portions of the heater assembly. As shown in the detailed image 901, in the respective heating areas of the sleeve 930, a fabric portion of the sleeve 927 may be disposed against the patient tissue/skin 2, wherein a patient-facing plastic layer 962a of the heater assembly may be positioned against the fabric layer 927 of the sleeve. The patient-facing layer 962a of the heater assembly may combine with an outward-facing plastic layer 962b, wherein certain components of the heater assembly are contained between the two plastic layers of the heater assembly in some embodiments. A heat-transfer medium 974, such as thermally-conductive gel or the like may be disposed adjacent to the plastic layer 962a. The thermal-conducting layer 974 may be disposed on a heating element/pad 960, which may be configured according to any of the embodiments disclosed herein and may advantageously be configured to radiate heat in the direction of the patient tissue 2, wherein such radiated heat is conducted through the thermally-conducting medium 974.

In some embodiments, a thermistor 968, or a plurality of thermistors, is/are disposed on the patient-facing surface of the heating pad/element 960. Although shown directly in contact with the patient-facing surface of the heating element/pad 960, it should be understood that the thermistor(s) 968 may be disposed in any position between the heating pad/element 960 and the patient tissue 2, whether internal to the heater assembly pouch or between the heater assembly pouch and the fabric layer 927, or between the fabric layer 927 and the patient tissue 2. In some embodiments, a backside (i.e., facing away from the patient) of the heating element/pad 960 may be thermally buffered by a thermal insulation layer 978, which may be disposed/present between the heating pad/element 916 and the outward-facing pouch layer 962b. In some embodiments, a fabric layer 929 is disposed on outermost portion of the heater assembly. That is, a fabric cover 929 or the like may be placed over the heater assembly when the heater assembly is coupled as desired to the sleeve 930.

FIGS. 9-2A, 9-2B, and 9-2C show cut-away views of a heating pad 975 of a heating assembly, the various views showing heating pad layers in accordance with one or more embodiments. FIG. 9-2D shows a cross-sectional view of a heating pad 975 of a heating assembly in accordance with one or more embodiments.

The heating pad 975 can comprise heat-transfer media 974, shown in FIG. 9-2C, which may not be present with respect to certain embodiments of heater assemblies in accordance with the present disclosure. For example, the heat-transfer media 974 may comprise thermal-/heat-transfer gel and/or other media configured to facilitate the transfer/conduction of thermal energy from the heater 960 through the patient-facing side of the heater assembly. In some embodiments, the heat-transfer media 974 comprises a thermal-transfer foam pad. In some embodiments, the heat transfer media 974 is disposed on, over, and/or under one or more thermistors 968, which may be used to determine temperature conditions at or near the heating element 960.

The heating element 960, which can be disposed between the thermal transfer layer 974 and a thermal insulating layer 978, may be any type of heater, such as a resistive radiating heater. For example, the heater 960 may comprise polyimide or other substrate material, which may be flexible or rigid in whole or in part. The thermal insulation layer/structure 978, which may comprise heat-insulating foam (e.g., relatively soft foam) and/or padding, may improve patient and/or physician comfort and/or reduce the risk of injury or damage to equipment from thermal energy originating in the heater 960. The insulation layer 978 can further advantageously present pressure on the heater 960 to push the heater 960 and thermal transfer layer 974 towards the patient to thereby improve contact and heat transfer to the patient.

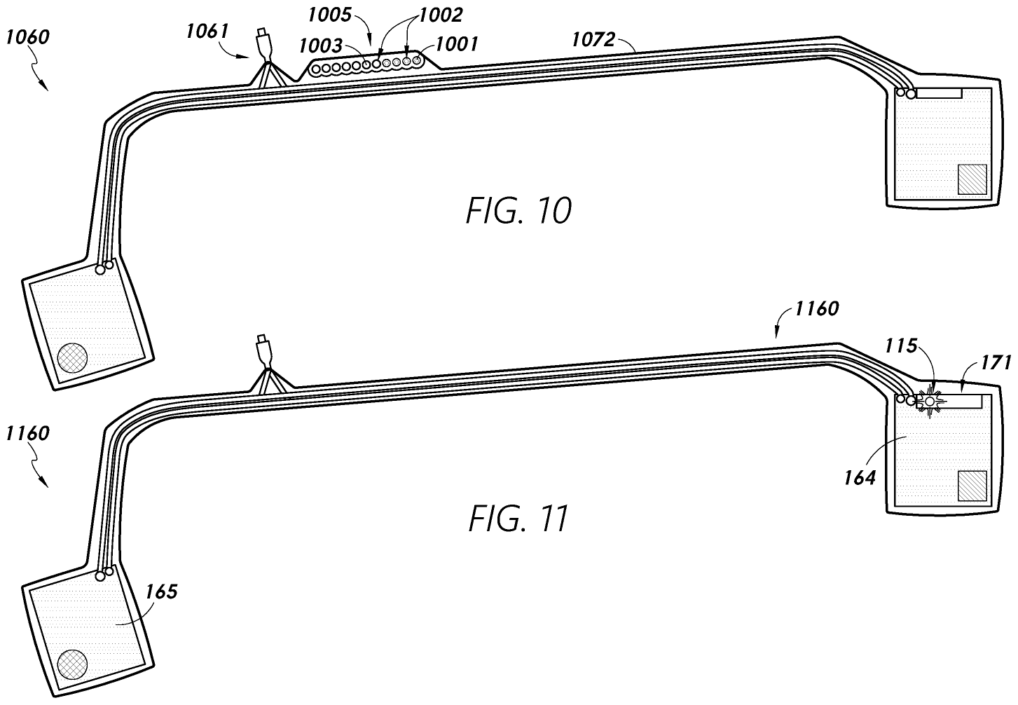

FIG. 10 shows a heater assembly including a mechanical usage/expiration indicator 1005 in according to one or more embodiments of the present disclosure. The usage indicator 1005 can be used to indicate to a user, such as a nurse or other technician, at what point the heater assembly 1060 has or will reach the end of its useful life according to some criteria. For example, the heater assembly 1060 may be considered a re-poseable device in that it may be used for multiple uses (e.g., multiple patients, multiple procedures, etc.), wherein the number of uses that may be used is limited in some manner. For example, in an example embodiment shown, the heater assembly 1060 may be suitable for 10 uses, after which it may be discarded.

The mechanical usage indicator 1005 may include one or more tabs, punch holes, or the like, wherein a user may mechanically punch a feature 1001 to thereby create an aperture/opening or other visual feature 1003 indicating a use count of the assembly 1060. That is, with each use, the user may punch one of the indicators 1001 to show a use count indicator 1003 in a used condition/state indicating that the use has occurred or will occur imminently. When the expiration indicator 1005 includes only used-condition counters 1003 (i.e., where each and every one of the use counts indicators old 1002 has been punch to a used condition/state 1003, the user may be notified or made aware that the heater assembly 1060 has reached the end of its useful life. In some embodiments, the mechanical usage indicator is associated with the sleeve member/article rather than the heater assembly. For example, with respect to embodiments including heater assemblies that are sewn into the sleeve without a pouch container around the heater assembly, it may be beneficial to incorporate the usage indicator (e.g., mechanical, or electrical as shown in FIG. 11) with other component(s) of the sleeve device, such as the sleeve fabric, electrical connector(s), strap(s), or the like.

FIG. 11 shows a heater assembly including an electrical usage/expiration indicator 115 in according to one or more embodiments of the present disclosure. The electrical usage indicator 115 may comprise one or more lights, such as LED lights or the like, which may be configured to flash and/or provide output (e.g. visual and/or audible) indicating a condition of the heater assembly 1160 with respect to the useful life and/or number of uses associated therewith. For example, the heater assembly 1160 may be configured to determine electrically when a use has been initiated or completed, such as by determining activation of one or more components of the heater assembly (e.g. heaters), wherein the heater assembly 1160 is configured to track a number of uses thereof and provide an indication to the user indicating the use condition of the heater assembly 1160. For example, in some implementations, the usage indicator 115 may flash and/or illuminate when the heater assembly 1160 is at or approaching the end of its useful life. In some embodiments, a color of the light 115 may indicate how close to the end of the useful life the heater assembly 1160 presently is.

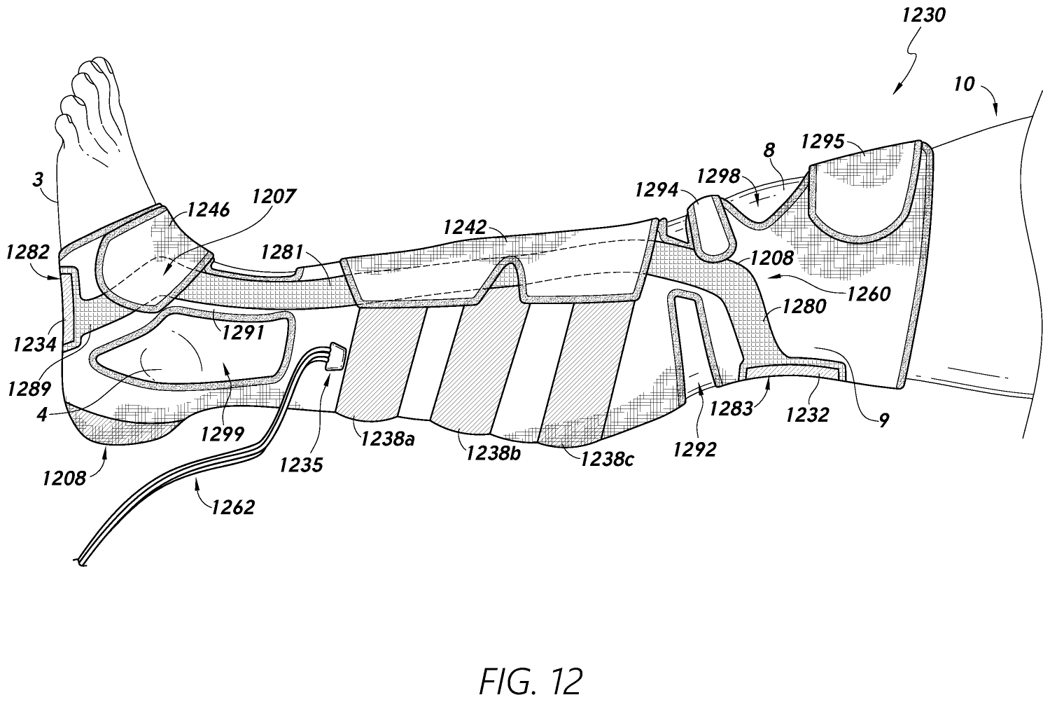

FIG. 12 illustrates a side view of a temperature-management sleeve system 1230 having a heater assembly 1260 secured thereto in accordance with one or more embodiments. As described in detail above, certain heater assemblies contemplated in connection with various embodiments of the present disclosure include two heater portions connected by a connector portion. For example. FIG. 12 shows a heater assembly 1260 including a first heater portion 1283 and a second heater portion 1282 connected by a connector portion 1281 that spans the distance between the two heater portions. For example, the connector portion 1281 may comprise an elongate portion of a pouch of the heater assembly 1260, which may have certain electrical wires and/or other components disposed therein. The shape, form, length, and/or other dimensions or characteristics of the connector portion 1281 may be designed in a manner as to conveniently and/or intuitively fit or run along a path associated with the sleeve 1230 in a manner that is consistent with the form of the sleeve 1230. For example, the connector portion 1281 of the heater assembly 1260 may include a first portion 1280 that extends from the popliteal fossa heating portion 1283 and runs along a first line/path, wherein the connector includes a bend 1208 that allows for redirection of the connector to follow along a path that is more aligned with the elongate dimension of the patient's limb 10 to span the distance between the heater portions of the heater assembly 1260.

The connector portion 1281 may include one or more additional bends, such as the band 1207, which may allow for redirection of the connector portion 1281 in the direction of the foot heater portion 1282 of the heater assembly 1260. That is, the illustrated connector portion 1281 includes one or more straight, curved, and/or bend portions, which represent merely examples of different routing features/characteristics of a connector portion of a heater assembly in accordance with aspects of the present disclosure. Therefore, it should be understood, that embodiments of the present disclosure may include heater assemblies having any number and/or configuration of straight, curved, and/or bend portions configured/designed to accommodate a suitable path along a wearable sleeve (e.g. for a leg or other limb) that allows for placement and/or coupling with/to the sleeve. For example, the straight, curved, and/or bend portions of the connector 1281 may be configured to pass around a gap or opening 1292 on a back of the leg portion of the sleeve 1230 and/or an opening 1298 on the front of the leg portion of the sleeve 1230 to allow for desirable flexibility for the sleeve 1230. Furthermore, the connector portion 1281 may be configured to follow a path along an ankle connector portion 1291 of the sleeve 1230, wherein the ankle connector portion 1291 is routed around an ankle opening 1299 which may further provide flexibility and/or comfort for the patient.

Connector portions of heater assemblies in accordance with aspects of the present disclosure may be configured to pass along and/or under certain physical couplings, such as straps or the like. For example, in the illustrated embodiment of FIG. 12, the connector portion 1281 passes at least partially under one or more straps 1242, 1246 of the sleeve 1230, which may allow for the straps to secure the connector portion 1281 in place and/or protect the connector portion from physical contact with items in the operating environment, which may otherwise dislodge the heater assembly 1260 or interfere therewith.

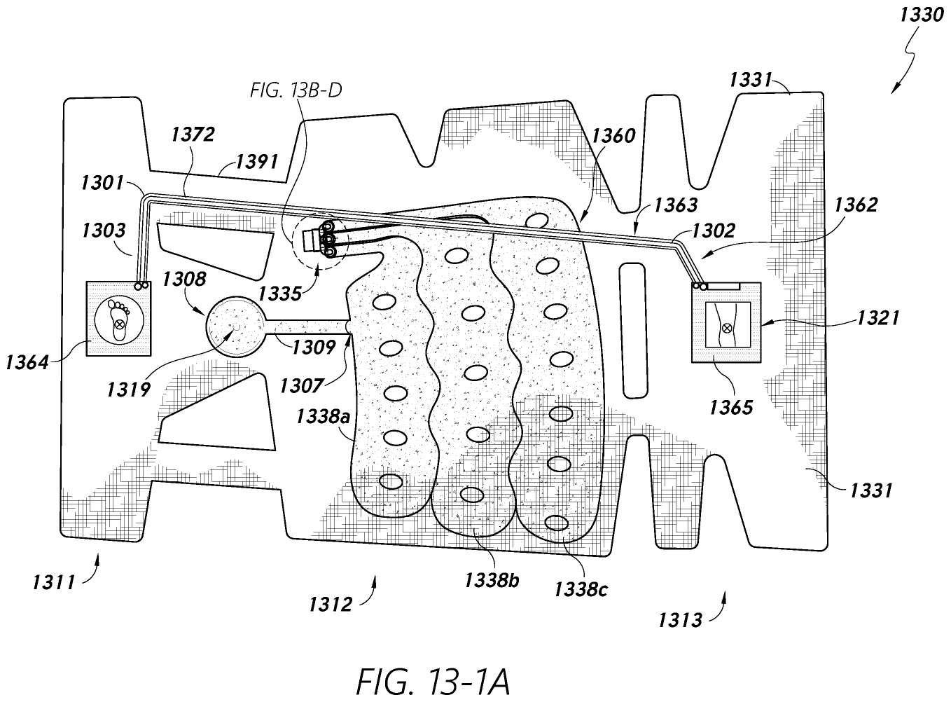

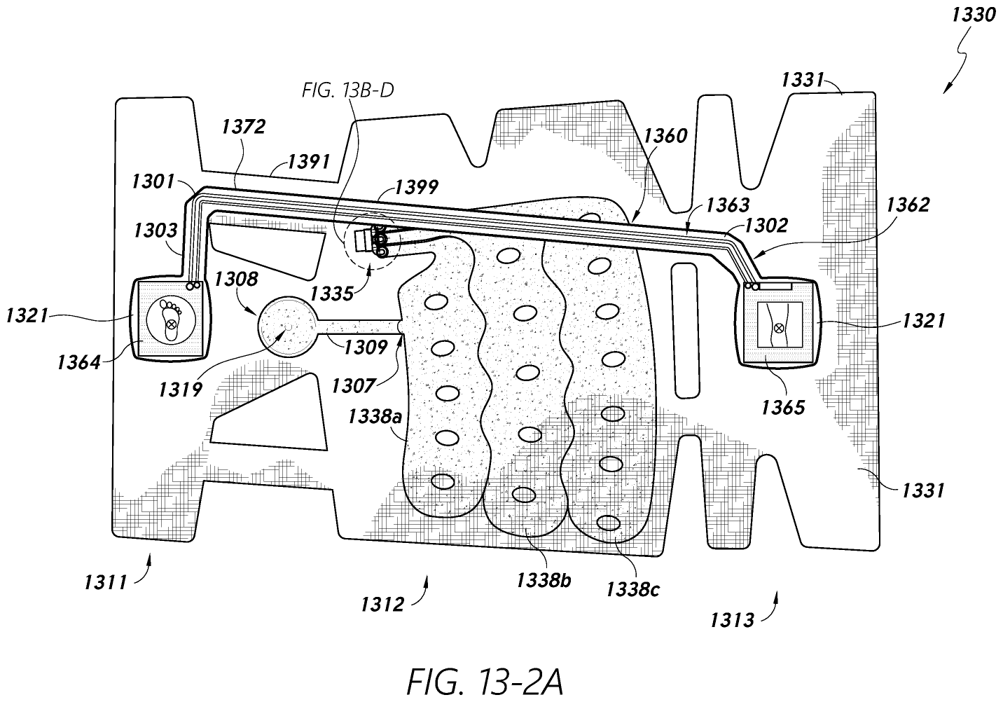

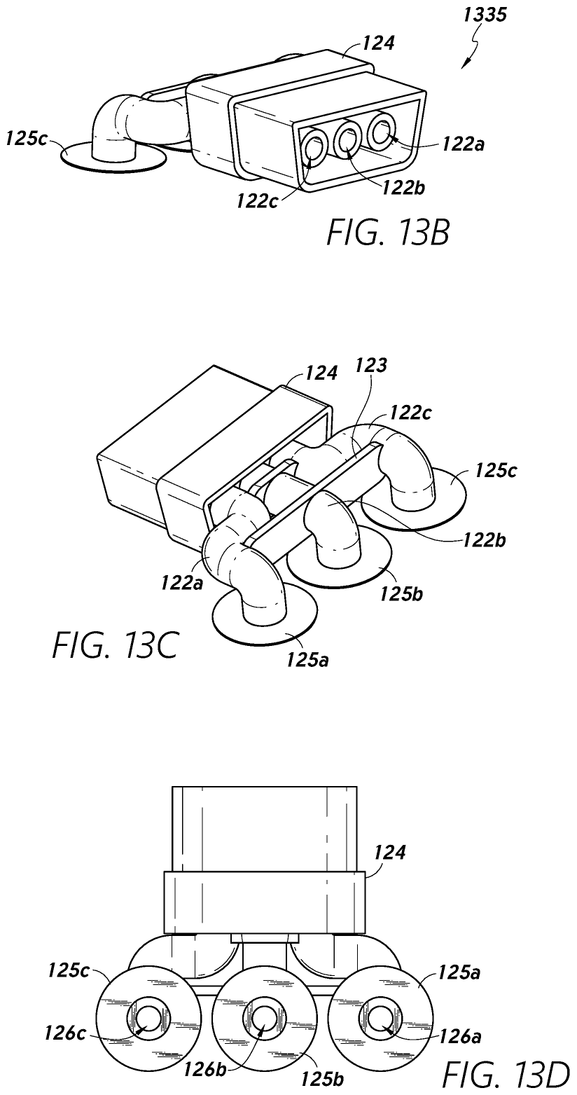

FIG. 13-1A illustrates a temperature-management system 1330 having a heater assembly 1360 placed thereon/therein in accordance with one or more embodiments. The temperature management system 1330 includes a sleeve structure/article 1331. The sleeve structure/article 1331 may or may not include a heater assembly delineation form or area, which may comprise one or more patches, stitches, routing paths or structures, or the like. Delineation of the heater assembly may be visually apparent by stitches in the sleeve 1331 that secure the heater assembly 1360 in place, such as between inner and outer layers of fabric of the sleeve 1331. For example, the heater assembly 1360 may be disposed on and/or in one or more portions of the sleeve structure 1331. In some examples, the heater assembly 1360 may rest and/or be secured on or to a heater assembly delineation form or area as described herein. In some implementations, the sleeve device/system 1330 may be a fully disposable product/design, wherein the heater assembly 1360 is integrated with the fabric assembly of the sleeve 1331 in some manner (e.g., stitching, welding, adhesive, etc.). The heater assembly 1360 in such implementations may generally not include an outer plastic/polymer cover/pouch, and may instead be permanently attached to the fabric of the sleeve 1331.

The temperature-management system 1330 can include a plurality of compression bladders 1338 associated with the calf portion 1312 of the sleeve 1331. For example, the compression bladders 1338 may include a lower bladder 1338a, an intermediate bladder 1338b, and an upper bladder 1338c. Although three bladders are shown in vertical arrangement with respect to the vertical orientation of a leg on which the sleeve 1331 may be placed, it should be understood that compression bladder assemblies in accordance with aspects of the present disclosure may include any number of compression bladders and/or any relative position and/or orientation. The temperature-management system 1330 of FIG. 13A advantageously includes an inflatable heel pad component 1308. Although the heel pad 1308 is shown as having a generally circular shape, it should be understood that heel pads in accordance with aspects of the present disclosure may have any suitable or desirable shape/form. In some embodiments, the heel pad 1308 is fluidly coupled to one or more of the compression bladders 1338 and/or to a fluid input connector 1335. For example, a fluid channel 1309 may be coupled between the heel pad 1308 and one of the compression bladders 1338 or other fluid channels, wherein a one-way valve 1307 allows for inflation of the heel pad 1308 when fluid pressure within one or more bladders or channels is greater than a certain threshold, wherein once the heel pad 1308 is inflated, the air within the heel pad 1308 may not be inclined to return through the valve 1307 and may be blocked by the one-way characteristics of the valve 1307. When the pressure within the channel 1309 and/or heel pad 1308 is greater than a certain threshold, air from within the bladder(s) 1308 and/or other channels upstream of the valve 1307 may generally not open and/or channel additional fluid into the channel 1309 and/or the heel pad 1308. In some embodiments, the heel pad 1308 includes a divot, hole, or other central depression feature 1319 configured to accommodate the apex/convex surface of the heel of the patient when placed on the heel pad 1308. The stitching securing the heater assembly 1360 to the sleeve 1331 may be implemented to avoid the area overlapping the bladder(s) so as to not affect the filling/sealing thereof.

The heater assembly 1360 may pass along one or more portions of the sleeve 1331, such as along a heel connector portion/path 1391. In some embodiments, the heater assembly connector portion 1362 may pass at least partially over a portion of the bladder/compression assembly 1338, as shown. As with certain other embodiments disclosed herein, the connector portion 1362 of the heater assembly 1360 may include one or more straight 1303, 1372 and/or bend 1301, 1302 sections, which may allow for the connector portion 1362 to traverse the area between the foot heating pad 1364 and the popliteal fossa heating pad 1365 on the sleeve 1331.

FIG. 13-2 is similar to FIG. 13-1, except that the heater assembly 1360 is shown in FIG. 13-2 as having a container pouch 1399 in which the heater assembly components (e.g., heaters/heater pads, wire connectors, etc.). With respect to any of the embodiments of sleeve and/or heater assemblies disclosed herein, such assemblies can include inflated pads or other features configured to press the heating pads/assembly against the popliteal fossa and/or sole of the foot in provide increased contact and/or heat transfer. For example. FIG. 13-2A shows heating pad pressing features 1321 disposed generally behind the heating pads 1364, 1365 when the heater assembly 1360 is placed on the sleeve 1330, wherein the pressing features 1321 are configured to exert force against the heating pads 1364, 1365 in the direction of the patient (i.e., out of the page with respect to the illustrated orientation of FIG. 13-2A) when the sleeve 1330 is secured to a limb of a patient.

FIGS. 13B-13D show views of a fluid connector 1335 of the temperature-management system of either of FIG. 13A-1 or 13A-2 in accordance with one or more embodiments. The fluid connector 1335 may include a plurality of fluid channels 122, each fluid channel being configured to be fluidly coupled to a separate bladder of a bladder compression assembly associated with the calf portion 1312 of the sleeve 1331. The connector 1335 may include flanges 125 associated with each of the fluid channels, which may be heat-sealed to the sleeve 1331 to prevent fluid leakage therethrough and provide a sound structural interface between the connector 1335 and the sleeve 1331. The connector 1335 may further include a consolidation structure 124 configured to contain the proximal portions of the fluid channels 122 such that a single fluid connector associated with the fluid source may be coupled to the structure 124 and provide fluid connection between the fluid source and the fluid channels 122.

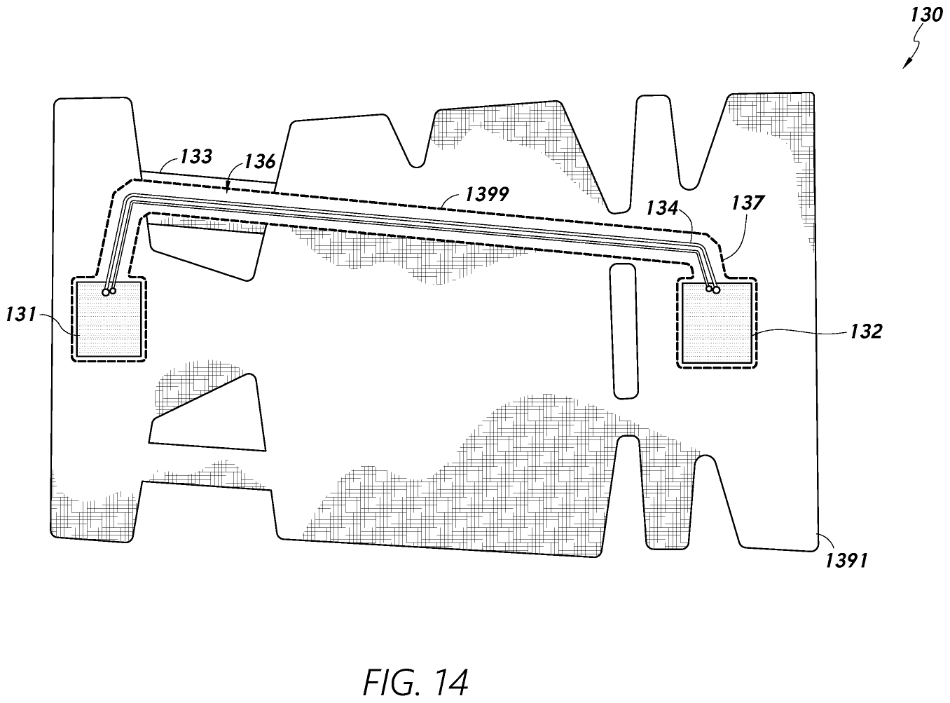

FIG. 14 shows a temperature-management sleeve 130 having a heater assembly 136 disposed and/or contained between layers of a sleeve article 1391 in accordance with one or more embodiments. The sleeve article 1391 may have any suitable or desirable form factor. For example, the sleeve 1391 may have a shape and/or form similar in certain respects to any of the embodiments disclosed herein. The heater assembly 136 may be a pouch-less heater assembly, as described in detail herein. The heater assembly 136 may be sewn and/or welded between inner and outer fabric layers of the sleeve 1391. The illustrated dashed lines 1399 represent sutures or other attachment mechanism/means used to attach the fabric layers of the sleeve article 1391 around the profile of the heater assembly 136 in a manner as to secure the heater assembly 136 in place within (or to an outer surface of) the sleeve 1391. The sutures or other attachment means may be placed/disposed around heater pads 131, 132 and/or a connector portion 134 of the heater assembly 136, as shown. The suturing/attachment of the fabric sleeve layers around the connector portion 134 of the heater assembly 136 may pass over one or more heater assembly bridge/passage features 133, as described above.

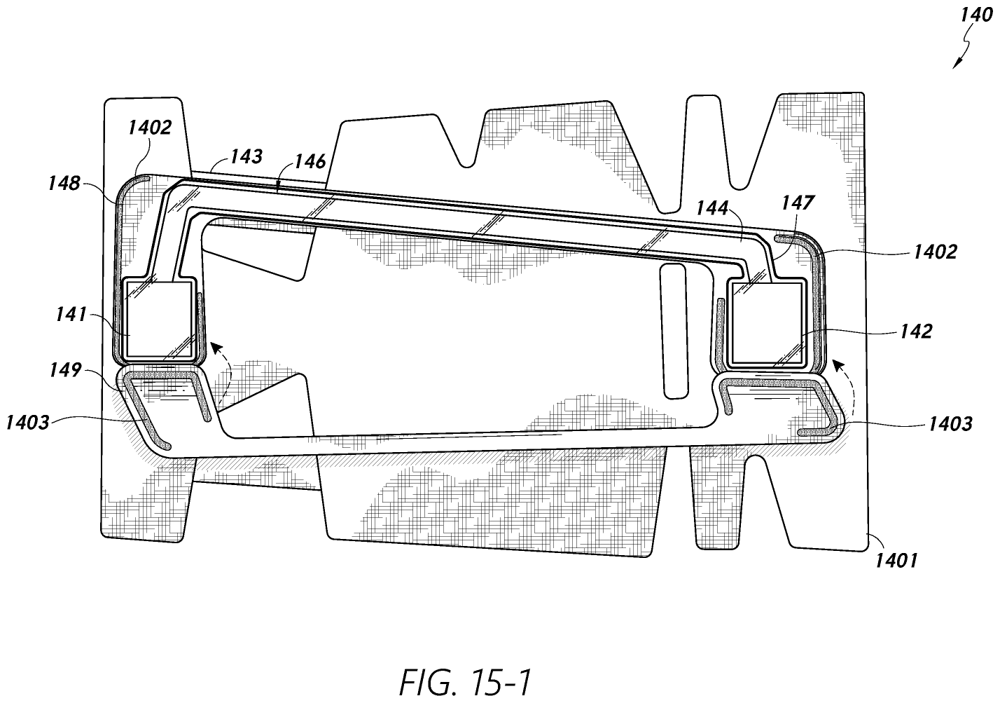

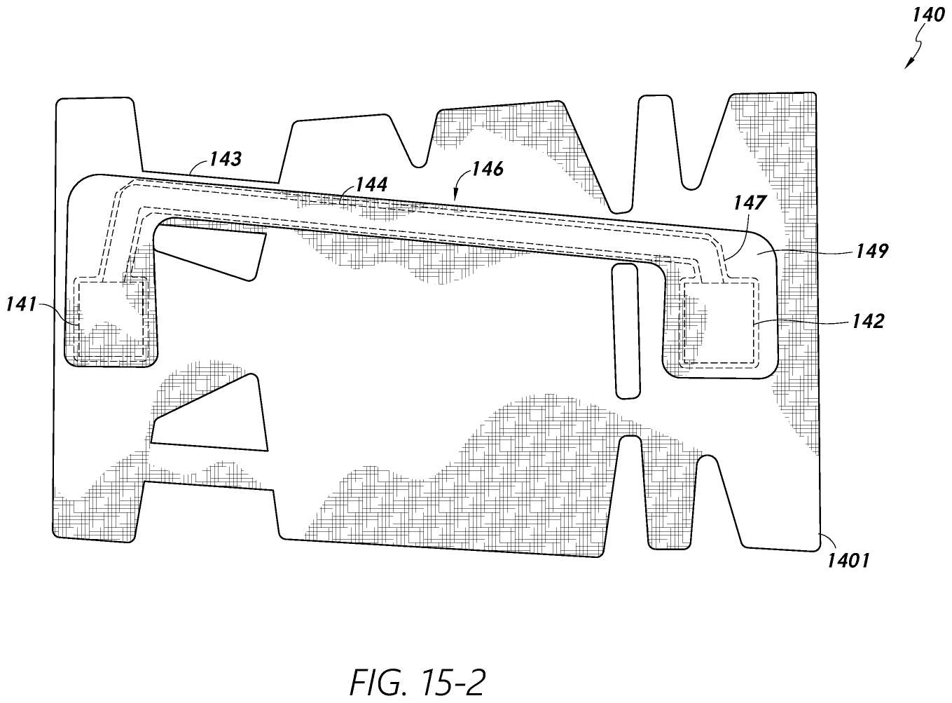

FIG. 15-1 shows a temperature-management sleeve 140 having a heater assembly cover 149 in accordance with one or more embodiments of the present disclosure. The sleeve 140 includes a heater assembly cover 149 which may be configured to be placed over at least a portion of the heater assembly 146 to thereby secure and/or position the heater assembly 146 to/on the sleeve 140. For example, the cover 149 may comprise fabric and/or other material. In some embodiments, the cover 149 includes certain attachment means 1403 for securing portion(s) thereof to other portion(s) of the sleeve 140. For example, such attachment means 1403 may comprise strips, band, and/or sections of Velcro or other attachment means. For example, in addition, or as an alternative, to Velcro, such attachment means 1403 may comprise one or more snaps, clips, buttons, wraps, folds, hooks, clasps, stitches/sutures, adhesives, adhesive contact surfaces, and/or other attachment means. In some embodiments, corresponding attachment means 1402 are associated with the body of the sleeve 140. The attachment means 1402 may be similar to the attachment means 1403 in some respects; any of the various types of attachment means discussed above with respect to the attachment means 1403 may be implemented with respect to the attachment means 1402.

In some embodiments, the attachment means 1402 is associated with a heater assembly delineation portion 148 of the sleeve 140. For example, the delineation portion 148 may comprise a form of fabric and/or other material on which the heater assembly 146 is disposed. For example, the delineation portion 148 may generally follow a similar path between the foot heat pad 141 and popliteal fossa heel pad 142 as the heater assembly, whether such path runs substantially on a straight path between the heating pads or on a more tortuous path/route as shown in FIG. 15-1, wherein the heater assembly 146 includes a connector portion 144 that has one or more bends therein to allow the connector portion 144 to run along a side portion of the sleeve 140 over at least a length thereof.

FIG. 15-2 shows the temperature-management sleeve 140 of FIG. 14 with the heater assembly cover 149 in a closed configuration in accordance with one or more embodiments. In the image of FIG. 15-2, the cover 149 it shown covering substantially the entire heater assembly 146. However, it should be understood, that certain cover and/or other coupling features associated with a sleeve in accordance with aspects of the present disclosure may cover only portion(s) of a heater assembly. For example, in some embodiments, cover portion(s) cover the heater pads of the heater assembly, while at least a portion of the connector portion of the heater assembly is not covered by the heater assembly cover.

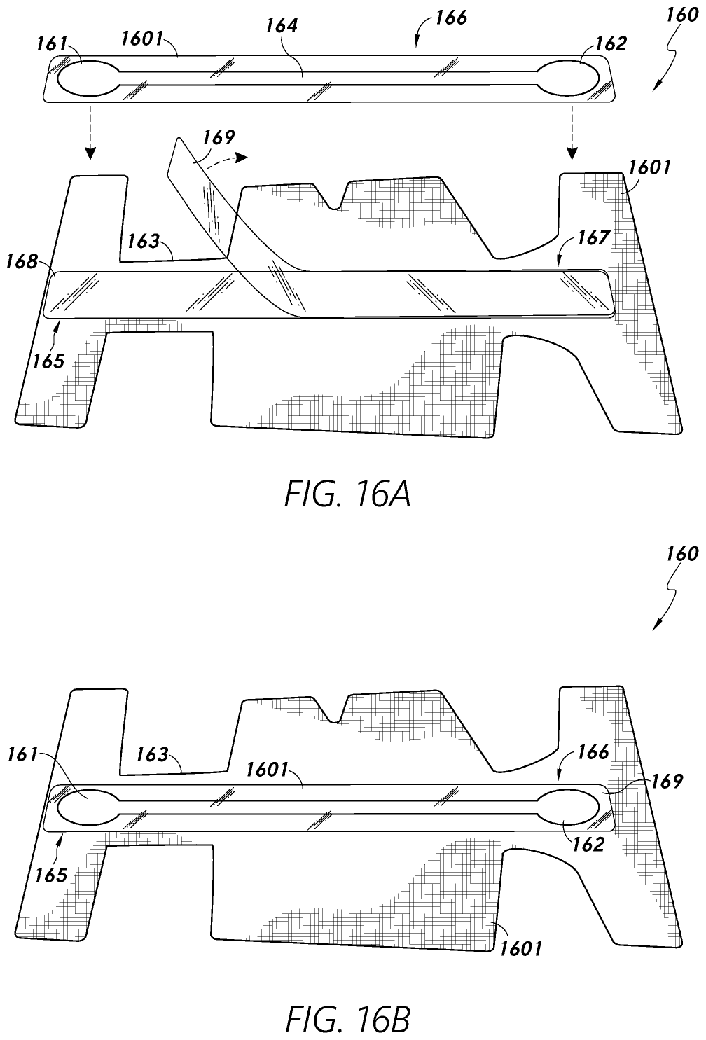

FIG. 16A shows a temperature-management sleeve 160 having an adhesive coupling feature 168 and a heater assembly 166 associated therewith in accordance with one or more embodiments of the present disclosure. As referenced above, certain adhesives may be implemented as attachment means for attaching heater assemblies to sleeve structures in accordance with aspects of the present disclosure. The sleeve structure 160 of FIG. 16A includes an adhesive surface 168 to which the pouch 1601 of the heater assembly 166 can be adhered to couple the heater assembly 166 to the sleeve 160. That is, the adhesive surface 168 may be physically coupled to the sleeve 160 in some manner, such as through use of one or more adhesives, stitches, and/or other attachment means in accordance with any of the various types of attachment means disclosed herein.

Prior to coupling of the heater assembly 166 to the sleeve structure 160, an adhesive covering 169 may be removed from over the adhesive surface 168, to thereby expose the adhesive surface 168 and allow for coupling therewith. For example, as shown in FIG. 16A, the adhesive covering 169 may be peeled back and/or off of the adhesive surface 168 to provide exposure therefore. In some embodiments, the heater assembly 166 is coupled to the sleeve structure/article 160 by stitching, welding, adhesive, or other attachment means, such that the heater assembly 166 is disposed/secured in place between fabric layers of the sleeve structure/article 160.

FIG. 16B shows the temperature-management sleeve 160 of FIG. 16A with the heater assembly 166 secured to the adhesive coupling feature 168 in accordance with one or more embodiments of the present disclosure. In the illustrated embodiment of FIGS. 16A and 16B, the adhesive surface 168 has a surface area similar to a surface area of the pouch 1601 of the heater assembly 166. However, it should be understood that adhesive surfaces in accordance with aspects of the present disclosure may have any size, shape, and/or form. For example, the adhesive surface 168, in some embodiments, may be limited to only covering certain portions of the heater assembly pouch 1601. For example, the adhesive surface 168 may be limited to the foot heater pad 165 and popliteal fossa 167 heater pad areas, wherein at least a portion of the connector portion 164 of the heater assembly 166 may not be adhered to the sleeve 160, but rather may be maintained in place through association/coupling with the heater pad portions 161, 162. Although FIGS. 16A and 16B show a heater assembly 166 that has a generally straight layout, wherein the heater assembly, when coupled to the sleeve 160, runs along a generally straight path between the foot portion 165 and popliteal fossa portion 167, it should be understood that the connector portion 164 of the heater assembly 166 may be formed and/or configured to be muted in any suitable or desirable path between the foot heater area 165 and popliteal fossa heater area 167 of the sleeve 160, as described in detail herein.

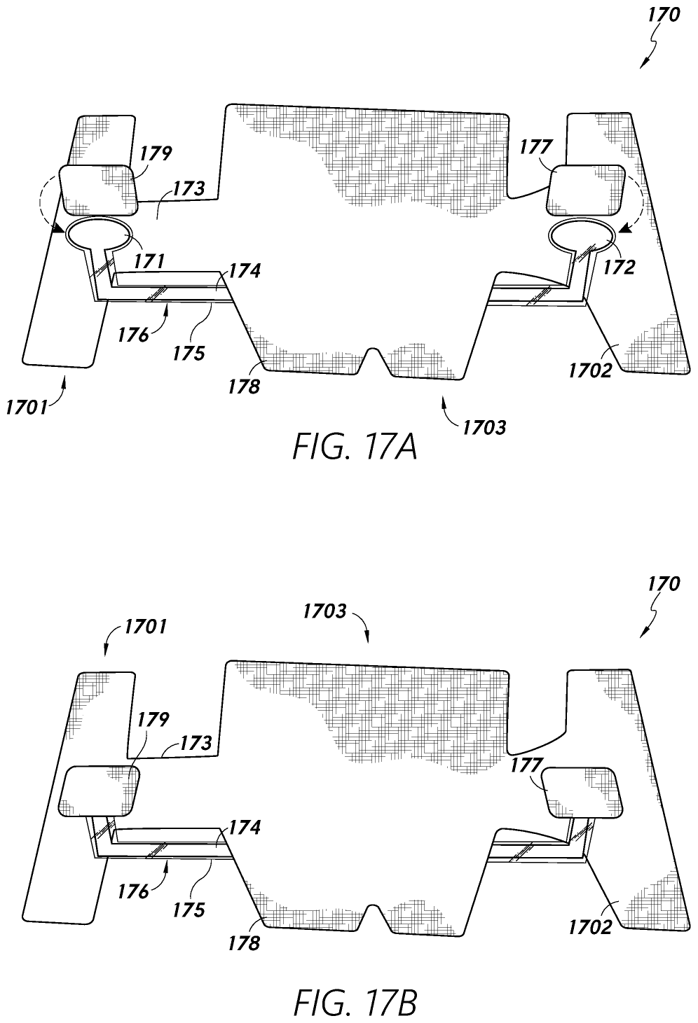

FIGS. 17A and 17B show a temperature-management sleeve 170 having a heater assembly 176 associated therewith with heater covers 177, 179 in open and closed configurations, respectively, in accordance with one or more embodiments of the present disclosure. The temperature-management sleeve 170 shown in FIGS. 17A and 17B may advantageously allow for routing of the connector portion 175 of the heater assembly 176 both on a patient facing side (i.e., the side visible and facing out of the page in FIGS. 17A and 17B) of the sleeve 170, as well as on an outer side (not visible in FIGS. 17A and 17B; i.e., the side facing generally away from the patient when the sleeve assembly 170 is disposed on a limb of the patient). By allowing the connector portion 175 to be routed outside of one or more portions of the sleeve 170, such portions may not contact the skin/anatomy of the patient directly in such regions, thereby potentially improving comfort and/or reducing the risk of damage to the heater assembly. Furthermore, the inside/outside routing of the heater assembly 176 and the connector portion 175 thereof can serve to secure the heater assembly in a secure position on/to the sleeve 170.

In the illustrated embodiment of FIGS. 17A and 17 B, the connector portion 175 of the heater assembly 176 can be passed along an outside portion of at least part of the calf portion 1703 of the sleeve. For example, the connector 175 may be configured to pass along an outside of a strap 178 or other physical coupling portion/component of the calf portion 1703 of the sleeve 170. Unlike the portion of the connector portion 175 that passes outside the calf portion 1703, the heating pad portions 171, 172 of the heater assembly 176 may be generally configured to be disposed on a patient-facing side of the sleeve 170, to thereby improve thermal transfer from the heating pads to the patient. The covers 179, 177 may be configured to cover over the heating pads 171, 172, respectively, to thereby prevent burn or other injury to the patient when the heating pads are activated. The covers 177, 179 may further serve to secure the heating assembly and/or heating pads thereof in place.

In some embodiments, the covers 179, 177 comprise fabric or other material designed to provide comfort for the patient at the heating pad contact areas of the sleeve 170. Any attachment means disclosed or contemplated herein may be used to secure the covers 179, 177 in the closed configuration shown in FIG. 17B, including Velcro, adhesive, snaps, classes, hooks, ties, sutures, or the like. The sleeve 170 may include certain recesses or other features in which the respective heating pad portions of the heater assembly 176 may be placed/disposed. For example, the sleeve 170 may include certain cutouts, depressions, indentations, apertures, cutaway layers, or the like corresponding to the respective shapes/forms of the heating pad portions 171, 172 of the heater assembly 176 to thereby secure such heating pads in place, guide proper placement of the heating pads, and/or serve any other purpose with respect to securing/placing the heating pad portions of the heater assembly 176. In some embodiments, the covers 179, 177 comprise heat-transfer medium, which may have characteristics in accordance with any heat transfer media described herein, such that the covers 179, 177 facilitate heat transfer between the heating pads 171, 172 and the patient's skin. For example, the covers 179, 177 can comprise high thermal conductivity fabric or other material (e.g., polymer). In some embodiments, thermally-conductive gel may be placed on/in the covers 179, 177.

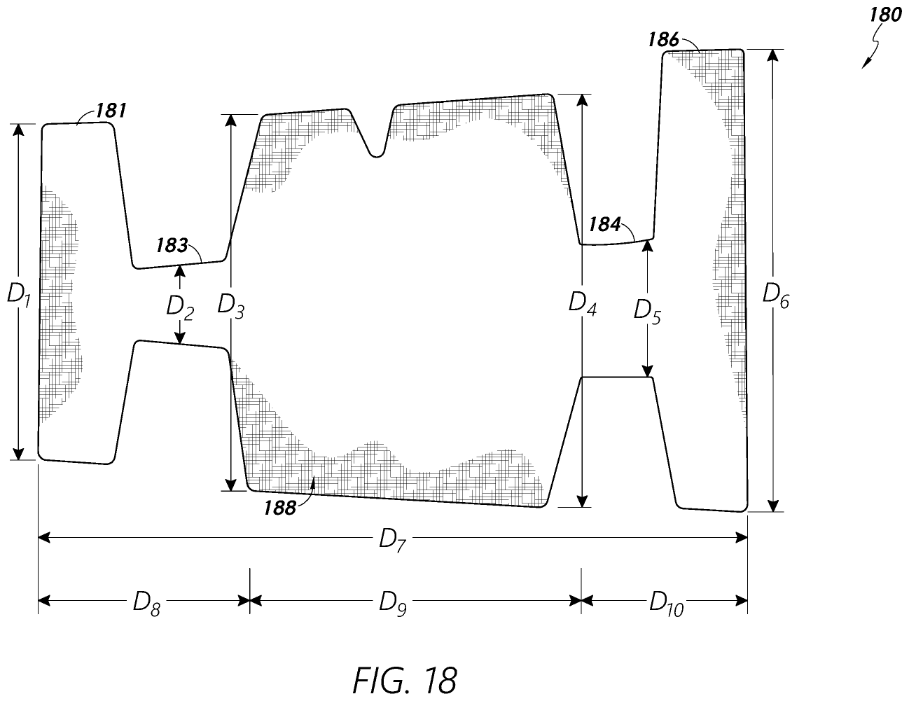

FIG. 18 shows a temperature-management sleeve 180 having certain dimensions in accordance with one or more embodiments. The various dimensions shown in FIG. 18 correspond to certain components, areas, and/or regions of the sleeve 180 with respect to one or more orientations or dimensions thereof. The various dimensions illustrated in FIG. 18 may take on certain values depending on whether the sleeve 180 is intended and/or considered as a large-sized, medium-sized, or small-sized sleeve, wherein each of such sizes is suitable for placement on a patient having a corresponding relative size with respect to the human population.

As with any of the embodiments of sleeves disclosed herein, the sleeve 180 may include a foot strap portion 181, which may be configured to be wrapped at least partially around a foot of the patient. The foot wrap portion 181 may have a width dimension D₁ that may be between approximately 12″-16″. According to some embodiments, a large-sized sleeve may include a foot wrap dimension D₁ of about 15″, whereas a medium-sized sleeve may include a foot wrap dimension D₁ of about 15″, whereas a small-sized sleeve may include a foot wrap dimension D₁ of about 13″.

Each of the remaining dimensions of the sleeve 180 of FIG. 18 are described below and include certain parentheticals with a list of four parameters, wherein such parameters include, in order, a range of values contemplated for such dimensions, as well as small, medium, and large size dimensions contemplated for such dimensions. Although specific values are listed, it should be understood that such values are approximations only and that actual value may be within approximately 10% of the recited values while falling within the scope of ranges of values contemplated explicitly with respect to FIG. 18.

Additional dimensions of the sleeve 180 include a width D₂ of an ankle connector portion 183 (2″-5″, 3″, 4″, 4″), a width D₃ of a calf portion 188 at a relatively narrow end or region thereof (16″-22″, 17″, 19″, 20″), a width D₄ of the calf portion 188 at a relatively wide area/portion thereof (18″-28″, 19″, 21″, 26″), a width D₅ of a popliteal fossa connector portion 184 (4″-6″, 4.5″, 5″, 5″), a width D₆ of a popliteal fossa wrap portion 186 (20″-29″, 22″, 25″, 27″), an overall length D₇ of the sleeve 180 (26″-37″, 28″, 32″, 35″), a length D₈ of the foot and ankle portions 181, 183 together (11″-16″, 12″, 13.5″, 14.5″), a length D₉ of the calf portion 188 (11″-16″, 12″, 13.5″, 14.5″), and a length D₁₀ of the popliteal fossa wrap 186 and connector 184 portions together (3″-7″, 4″, 5″, 6″). In some embodiments, the foot and ankle portions 181, 183 may be detachable from the calf portion 188. Furthermore, the popliteal fossa 186 and connector 184 portions may be detachable from the calf portion 188 in some manner.

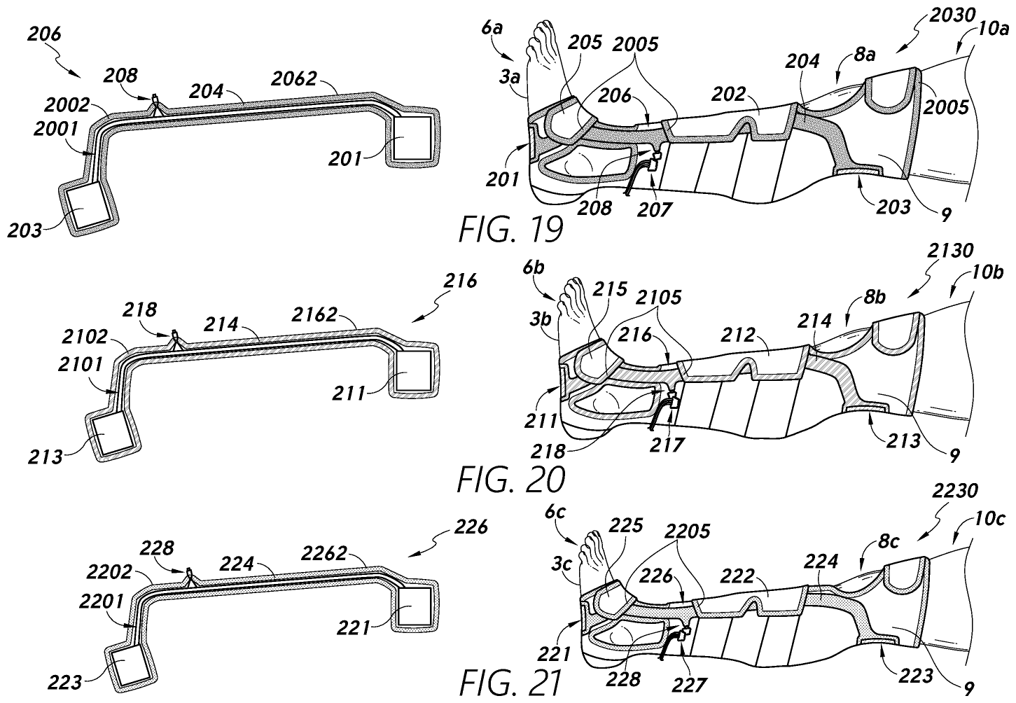

FIGS. 19-21 shows color-coordinated temperature-management sleeves and heater assemblies in accordance with one or more embodiments. Generally, the temperature-management sleeve 2030 and associated heater assembly 206 of FIG. 19 may correspond to a relatively large-sized implementation of a temperature-management sleeve and heater assembly. That is, the sleeve 2030 may be sized, dimension, and/or configured to be disposed about the lower limb of a relatively large human patient, wherein the heater assembly 206 is likewise dimensioned with dimensions that allow for placement on the relatively large sleeve 2030. Therefore, the connector portion 204 of the heater assembly 206 may be relatively long compared to smaller-sized sleeves and temperature-management systems/assemblies.

The heater assembly 206 may include certain color-based and/or other types of visual indicators that indicate that the heater assembly 206 is intended for use with a relatively large sleeve (i.e., the sleeve 2030). For example, as shown, certain portions of the pouch 2062 of the heater assembly 206 may include colored features 2002 and/or other visual markings/indicators indicating a size aspect/attribute of the heater assembly 206. The sleeve 2030 may further include certain corresponding color or other visual indicators/markings 2005 indicating the relative size of the sleeve 2030. For example, the color features 2005 may indicate that the sleeve 2030 is a relatively large-sized sleeve.

The heater assembly 206 may be configured to be physically secured or coupled to the sleeve 2030, as shown in FIG. 19, wherein the foot heating pad portion 203 thereof may be positioned/disposed on or proximate to the sole 3 of the foot 6 of the patient, wherein the popliteal fossa heater pad 201 is configured to be placed/disposed on or near a popliteal fossa region 9 of the patient's limb 10a, as shown. The connector portion 204 of the heater assembly 206 may be configured to be routed between the foot heating pad 203 and the popliteal fossa heating pad 201 along one or more portions of the sleeve 2030, as shown. The heater assembly 206 may include an electrical connector 208 configured to be electrically coupled to a connector 207 associated with the sleeve 2030, as shown.

The temperature-management sleeve 2130 and associated heater assembly 216 of FIG. 20 may correspond to a medium-sized implementation of a temperature-management sleeve and heater assembly. That is, the sleeve 2130 may be sized, dimension, and/or configured to be disposed about the lower limb of a medium-sized human patient, wherein the heater assembly 216 is likewise dimensioned with dimensions that allow for placement on the medium-sized sleeve 2130. Therefore, the connector portion 214 of the heater assembly 216 may be relatively long compared to smaller-sized sleeves and temperature-management systems/assemblies, and relatively short compared to larger-sized sleeves and temperature-management systems/assemblies.

The heater assembly 216 may include certain color-based and/or other types of visual indicators that indicate that the heater assembly 216 is intended for use with a medium-sized sleeve (i.e., the sleeve 2130). For example, as shown, certain portions of the pouch 2162 of the heater assembly 216 may include colored features 2102 and/or other visual markings/indicators indicating a size aspect/attribute of the heater assembly 216. The sleeve 2130 may further include certain corresponding color or other visual indicators/markings 2105 indicating the relative size of the sleeve 2130. For example, the color features 2105 may indicate that the sleeve 2130 is a medium-sized sleeve.

The heater assembly 216 may be configured to be physically secured or coupled to the sleeve 2130, as shown in FIG. 20, wherein the foot heating pad portion 213 thereof may be positioned/disposed on or proximate to the sole 3 of the foot 6 of the patient, wherein the popliteal fossa heater pad 211 is configured to be placed/disposed on or near a popliteal fossa region 9 of the patient's limb 10b, as shown. The connector portion 214 of the heater assembly 216 may be configured to be routed between the foot heating pad 213 and the popliteal fossa heating pad 211 along one or more portions of the sleeve 2130, as shown. The heater assembly 216 may include an electrical connector 218 configured to be electrically coupled to a connector 217 associated with the sleeve 2130, as shown.

The temperature-management sleeve 2230 and associated heater assembly 226 of FIG. 21 may correspond to a relatively small-sized implementation of a temperature-management sleeve and heater assembly. That is, the sleeve 2230 may be sized, dimension, and/or configured to be disposed about the lower limb of a relatively small human patient, wherein the heater assembly 226 is likewise dimensioned with dimensions that allow for placement on the relatively small sleeve 2230. Therefore, the connector portion 224 of the heater assembly 226 may be relatively short compared to larger-sized sleeves and temperature-management systems/assemblies.

The heater assembly 226 may include certain color-based and/or other types of visual indicators that indicate that the heater assembly 226 is intended for use with a relatively small sleeve (i.e., the sleeve 2230). For example, as shown, certain portions of the pouch 2262 of the heater assembly 226 may include colored features 2202 and/or other visual markings/indicators indicating a size aspect/attribute of the heater assembly 226. The sleeve 2230 may further include certain corresponding color or other visual indicators/markings 2205 indicating the relative size of the sleeve 2230. For example, the color features 2205 may indicate that the sleeve 2230 is a relatively small-sized sleeve.

The heater assembly 226 may be configured to be physically secured or coupled to the sleeve 2230, as shown in FIG. 21, wherein the foot heating pad portion 223 thereof may be positioned/disposed on or proximate to the sole 3 of the foot 6 of the patient, wherein the popliteal fossa heater pad 221 is configured to be placed/disposed on or near a popliteal fossa region 9 of the patient's limb 10c, as shown. The connector portion 224 of the heater assembly 226 may be configured to be routed between the foot heating pad 223 and the popliteal fossa heating pad 221 along one or more portions of the sleeve 2230, as shown. The heater assembly 226 may include an electrical connector 228 configured to be electrically coupled to a connector 227 associated with the sleeve 2230, as shown.

### Temperature-Management Processes

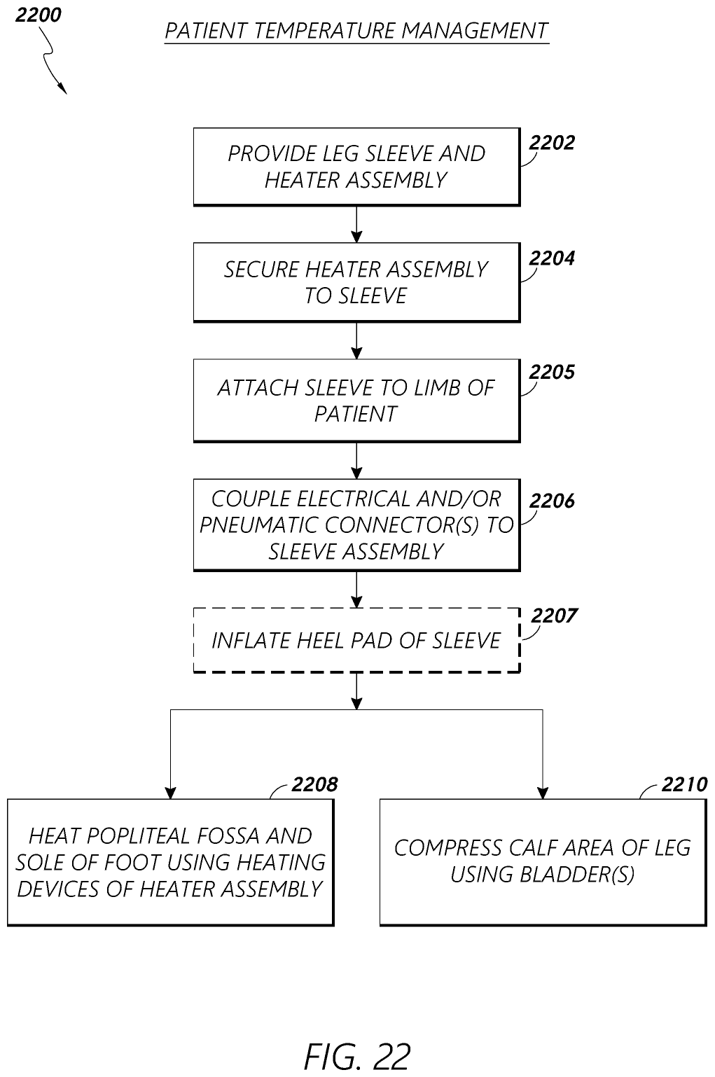

FIG. 22 is a flow diagram illustrating a process 2200 for managing patient temperature in accordance with one or more embodiments. At block 2202, the process 2200 involves providing a leg sleeve and heater assembly as described in accordance with various embodiments disclosed herein. For example, in connection with the operation(s) associated with block 2202, the process 2200 may involve accessing/providing a sleeve structure and a physically separate heater assembly configured to be physically secured/coupled to the sleeve.

At block 2204, the process 2200 involves securing/attaching the heater assembly to the sleeve. For example, attaching the heater assembly to the sleeve may involve engaging or attaching one or more straps, or other attachment means as described herein on or around one or more portions of the heater assembly. For example, one or more covers or other structures of the sleeve may be placed over one or more portions of the heater assembly and secured in a manner as to physically couple the heater assembly to the sleeve. In some embodiments, a portion of the heater assembly may be secured to the sleeve on a patient-facing side thereof, whereas one or more other portions of the heater assembly may be disposed outside of the heater assembly when the sleeve is attached to the limb of the patient.

In some implementations, the heater assembly may be stitched, welded, adhered, or otherwise attached/secured between fabric layers of the sleeve. For example, the heater assembly may be a pouch-less heater assembly (e.g., for a disposable embodiment of a temperature management sleeve).

At block 2205, the process 2200 involves physically attaching the sleeve to a limb of a patient. For example, the attaching the sleeve may involve fastening one or more straps or other physical attachment means/mechanism, as described in detail herein. Attaching may involve fastening attachment means associated with a foot portion, a calf portion, and a popliteal fossa portion of the sleeve.

At block 2206, the process 2200 involves coupling one or more electrical and/or pneumatic connectors to the sleeve assembly, wherein such connectors may be provided from one or more supply units, such as a control unit providing air compression and/or electrical resources for the sleeve assembly. The process 2200 may further involve inflating an inflatable heel pad associated with a heel portion of the sleeve, as shown as block 2207. For example, inflation of the heel pad may be implemented using air received over one or more of the connector(s) coupled in connection with block 2206.

In some embodiments, the sleeve assembly, with the heater assembly attached thereto and/or incorporated therewith, can include compression and heating functionality. That is, the sleeve structure may include one or more compression bladders, which may be implemented in accordance with a sequential compression process or mechanism as disclosed herein. Therefore, with the sleeve assembly disposed about the leg of the patient, the process 2200 may involve concurrently and/or over a common period of time, heating the popliteal fossa and sole of the foot using heating pad/devices of the heater assembly, as shown at block 2208, and applying compression to the calf area of the leg of the patient using one or more inflatable compression bladders of the sleeve structure, as shown at block 2210.

### Temperature-Management Sleeve Size Adjustability

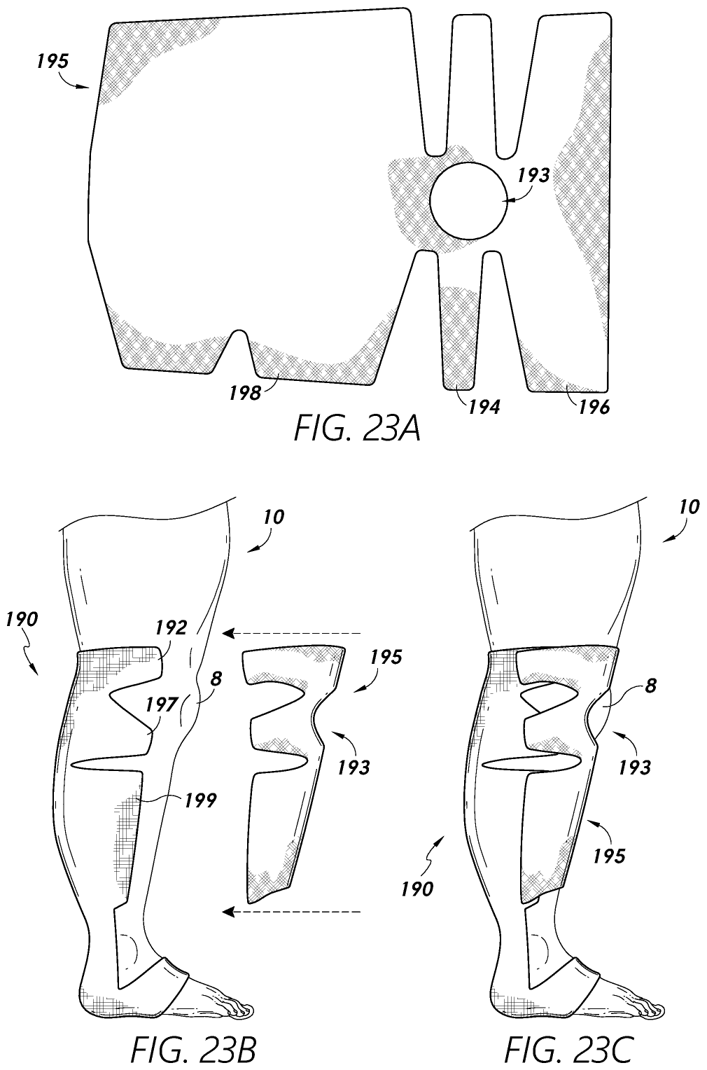

The various temperature-management sleeve assemblies disclosed herein may be adjustable in various manners to accommodate different sizes of patient limbs/legs. FIG. 23A shows a temperature-management sleeve extension component 195 in accordance with one or more embodiments of the present disclosure. The sleeve extension component 195 may be configured and/or shaped/dimensioned to be placed over the shin and/or other portions of the patient's leg to provide extension for a sleeve to which the extension component 195 is configured to be attached. For example, the sleeve extension 195 may include an opening/aperture 193, which may be configured to be placed over the knee of the patient, whereas one or more strap portions 198, 194, 196 of the extension component 195 may be designed and/or configured to be physically coupled to corresponding attachment means/features of a temperature-management sleeve structure in accordance with one or more embodiments of the present disclosure.

FIGS. 23B and 23C shows a temperature-management sleeve 190 disposed on a lower limb of a patient with the sleeve extension component 195 of FIG. 23A in a detached and attached configuration, respectively, in accordance with one or more embodiments. As shown in FIGS. 23B and 23C, the extension component 195 may be placed over the leg 10 of the patient, such that the opening 193 is disposed about the knee 8 of the patient's limb 10. The coupling of the extension component 195 to the sleeve 190 can advantageously provide an extended diameter/width dimension/aspect of the sleeve 190, such that it may be coupled around a leg that is of a size such that the side straps 199, 192, 197 of the sleeve 190 do not reach fully around the circumference of the limb in one or more portions of the sleeve 190 and/or leg 10.

### Temperature-Management Sleeve Assemblies

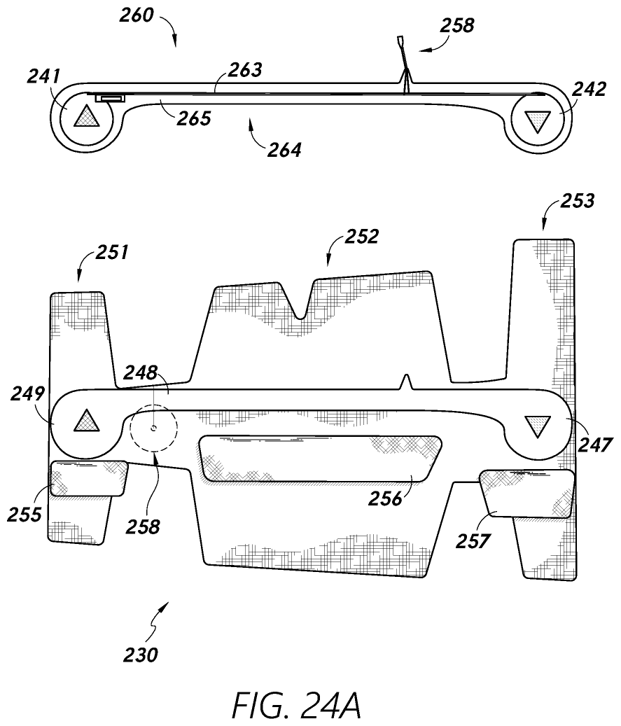

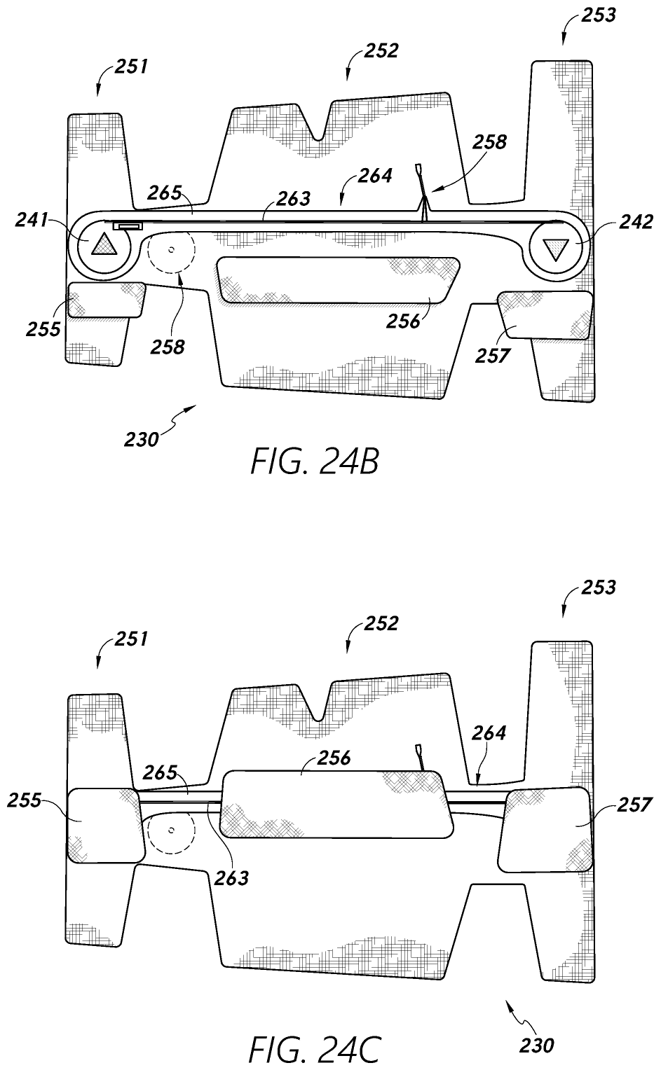

FIGS. 24A-24C illustrate a wearable sleeve 230 and heater assembly 260 in various configurations and/or coupling states in accordance with aspects of the present disclosure. The visible side of the sleeve 230 shown in FIGS. 24A-24C may be a patient-facing side, or alternatively may be a side of the sleeve that faces away from the patient when the sleeve 230 is disposed on a limb of the patient.

Although certain embodiments are disclosed herein in which heater assemblies including connector portions that are routed in relatively tortuous paths along segments of the coupled sleeve, the embodiments shown in FIGS. 24A-24C include a heater assembly 260 and associated structure of the sleeve 230 that allows for the connector portion 264 of the heater assembly 260 to take a relatively straight path between a foot heater area 249 and a popliteal fossa heater area 247 of the sleeve 230. For example, the path 248 between such seating areas may be substantially straight/linear.

In the embodiment of FIGS. 24A-24C, the heater assembly 260 may be placed, as shown in FIG. 24B, generally along a central band/path of the sleeve 230. For example, the sleeve 230 may include a heater assembly delineation feature 248, which may comprise one or more layers of material and/or other stitches, indentations, raised features, or other features or indicators corresponding to the profile of the heater assembly 260 with respect to the desired or intended positioning of the heater assembly 260 on the sleeve 230. In some embodiments, the sleeve 230 includes an inflatable, or non-inflatable, heel pad 258, which may generally be disposed in an area of the sleeve between the foot portion 251 and the calf portion 252.

The sleeve 230 may further include one or more cover portions 255, 256, 257, which may be configured to be placed at least partially over one or more portions of the heater assembly 260 when the heater assembly 260 is disposed in its intended position on the sleeve 230. For example, in some embodiments, the sleeve 230 may include a foot heater cover portion 255 configured to be placed over the foot heater 241 of the heater assembly 260, as shown in FIG. 24C. The sleeve 230 may further comprise a connector cover portion 256, which may be generally associated with the calf portion 252 of the sleeve 230 and may be configured to cover over at least a portion of the connector 264 of the heater assembly 260. The sleeve 230 may further comprise a popliteal fossa heater cover portion 257, which may be configured to be placed at least partially over the popliteal fossa heating pad 242 of the heater assembly 260 when the heater assembly 260 is coupled to the sleeve 230.

Certain embodiments of heater assemblies disclosed herein and shown in the various figures accompanying the present disclosure include wires and/or connector portions between the respective heating pads of the heater assembly that run generally along a central path between the heating pads. That is, with respect to a center of mass of the heating pads of the heater assembly, the wires may be coupled to the respective heating pads in an area generally aligned with the centers of the heating pads and/or the connector may run along a path that is generally aligned with the centers of mass of the heating pads. Alternatively, as shown in FIGS. 24A-24C, the wires 263 may be offset from the respective centers of the areas of the heating pads 241, 242. For example, the wires may be coupled to the respective heating pads at or near a common side of the heating pads, as shown. In some embodiments, the pouch 265 generally outlines the offset connector portion 264, as shown. The offset configuration of the connector 264 relative to the center of the heating pads can allow for the connector 264 to be routed at least partly around the inflatable heel pad 258, as shown in FIG. 24B. Although shown with cover portions, it should be understood that, as with any other embodiment disclosed herein, the heater assembly 260 may be pouch-less, and may be secured between inner and outer fabric layers of the sleeve 230, such as through stitching, welding, adhering, or other attachment means, such as around a profile of the heater assembly 260.

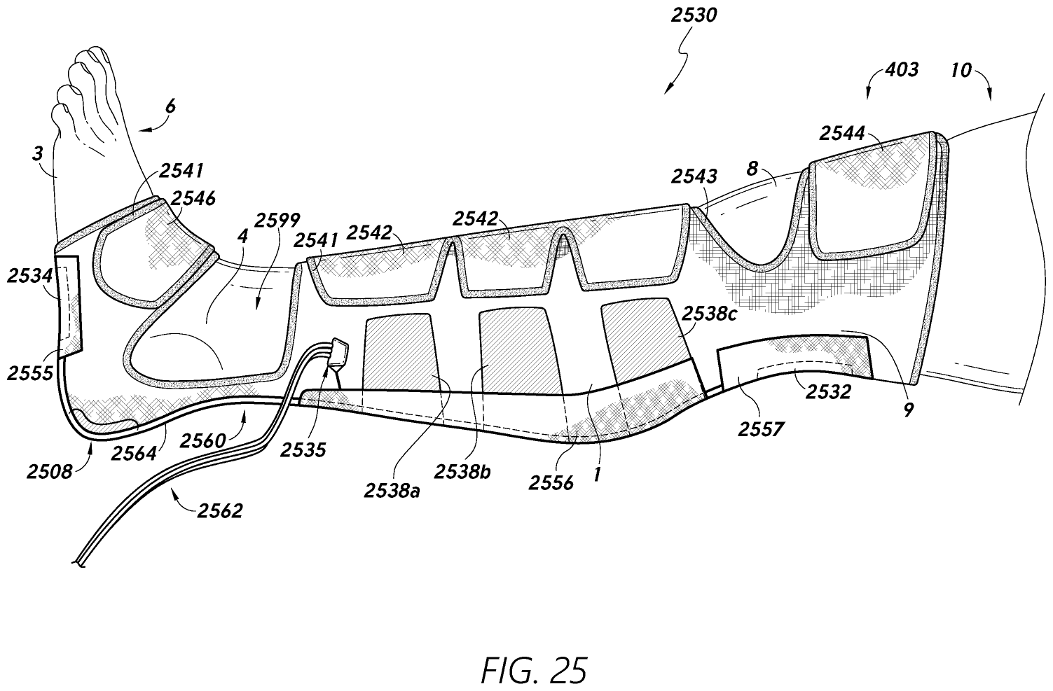

FIG. 25 shows a temperature-management sleeve 2530 disposed on a limb 10 of a patient, wherein the sleeve 2530 includes separate heater pad and heater assembly connector cover portions 2555, 2556, 2557, in a similar manner as shown in FIGS. 24A-24C. The sleeve assembly 2530 includes a heater assembly 2560 having a foot heater 2534 and a popliteal fossa heater 2532 connected by a connector portion 2564 that spans a portion of the sleeve 2530 between the foot 6 and the popliteal fossa 9. The sleeve 2530 may include a first cover 2555 that is configured to cover over the foot heater 2534, a second cover 2557 configured to cover over the popliteal fossa heater 2532, and a calf cover portion 2556 configured to cover over at least a portion of the connector 2564 of the heater assembly 2560 in the area of the calf of the patient.

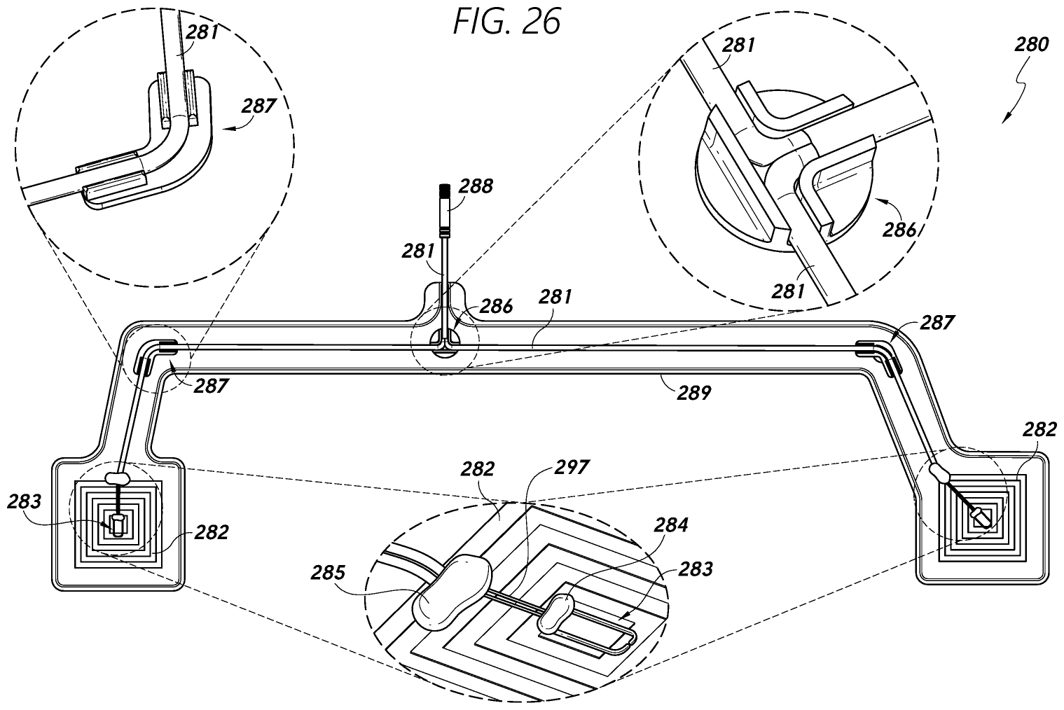

FIG. 26 shows a cut-away view of a heater assembly 280 in accordance with one or more embodiments. The heater assembly 280 includes certain wiring (e.g., electrical) 281, which may provide electrical current to the heating pads 282 to power the heating thereof. For example, the heating pads 282 may be configured to conduct electrical current to produce resistive heating effects, as described in detail herein. The wiring 281 may terminate at the heating pads 282, wherein certain electrically- and/or thermally-insulating sealant, adhesive, or the like 285, 284, which may be provided to secure and/or couple the wiring and/or components associated therewith to the respective component(s) of the heating pads 282. For example, the sealant 285, 284 may be implemented to cover otherwise exposed wiring to prevent damage thereto or other undesirable electrical interaction/effects.

The wiring 281 of the heater assembly 280 may be disposed within a polymer pouch 289, or may be independent of any pouch. That is, the heater assembly 280 may be contained at least partially within a pouch 289 or may be a pouch-less embodiment of a heater assembly, as described in detail herein. The configuration of the wiring 281 may be facilitated through the use of one or more wire-management structures/clips 286, 287. For example, in some embodiments, such features 286, 287 may comprise injection-molded clips configured to physically hold/secure the wiring 281 in place during assembly and/or after assembly/manufacturing. In some embodiments, such wire-management clips may include certain channels or grooves into which the wiring 281 may be placed, wherein such channels/grooves orient and/or direct the wiring 281 in a desired orientation/direction. For example, the wire-management clips 286, 287 can be implemented in areas of the heater assembly 280 where the wiring 281 is bent and/or changes direction, such as at the 90° bends associated with the central clip 286, wherein the wiring 281 may be bent/channeled in opposite directions, which may present a 90° directional turn from the angle of the wiring 281 emanating from the connector 288, as shown. In addition, one or more elbow clips 287 may be implemented to bend the wiring 281 at a certain angle(s) towards a respective heating pad 282, as shown. In some embodiments, the heater assembly 280 may comprise two elbow clips 287, wherein each elbow clip 287 may be configured to bend the wiring 281 at a different angle to accommodate the particular layout/configuration of the wiring 281 of the heater assembly 280.

The electrical wiring 281 may further include certain wiring 297 for communicating power and/or other signals to/from a thermistor 283 or other temperature sensor device/element. The wiring 297 may be sealed and/or otherwise coupled to the heater pad 282 by the sealant/adhesive 284. The thermistor wiring 297 may be part of the wiring 281, but may be separate from wiring configured to power the heating pad 282.

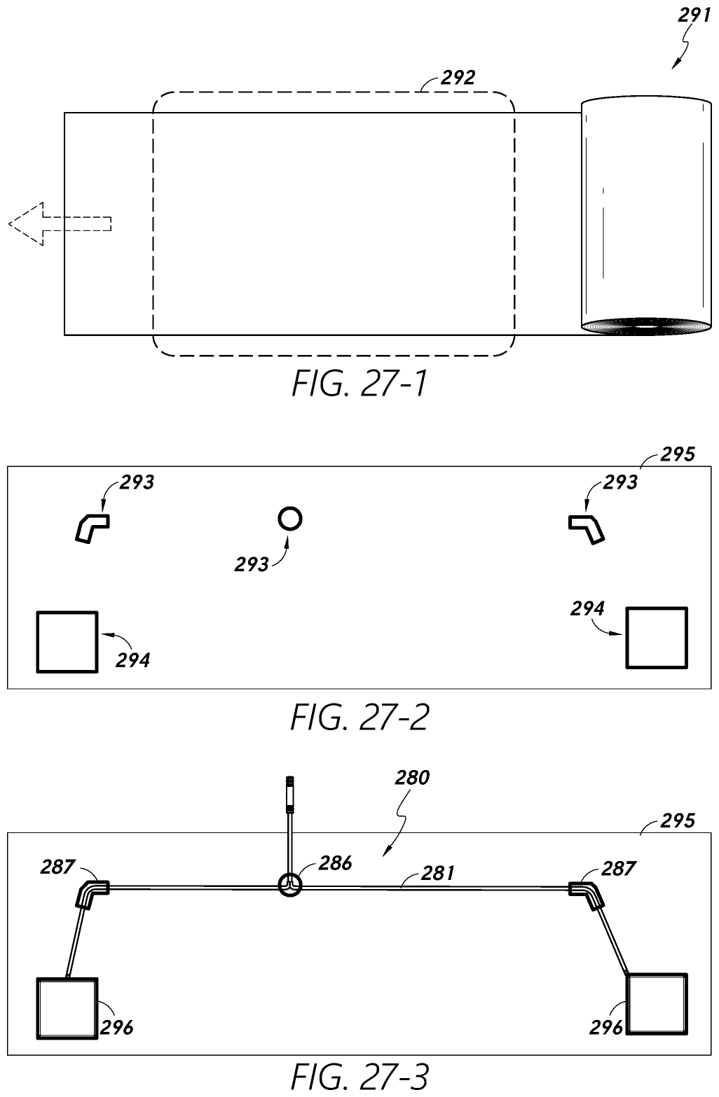

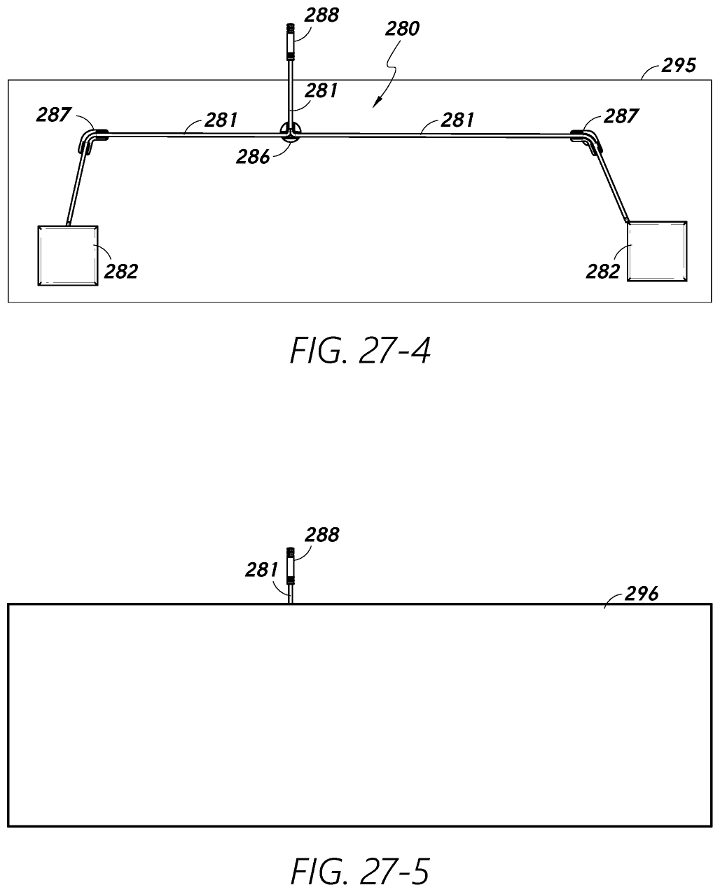

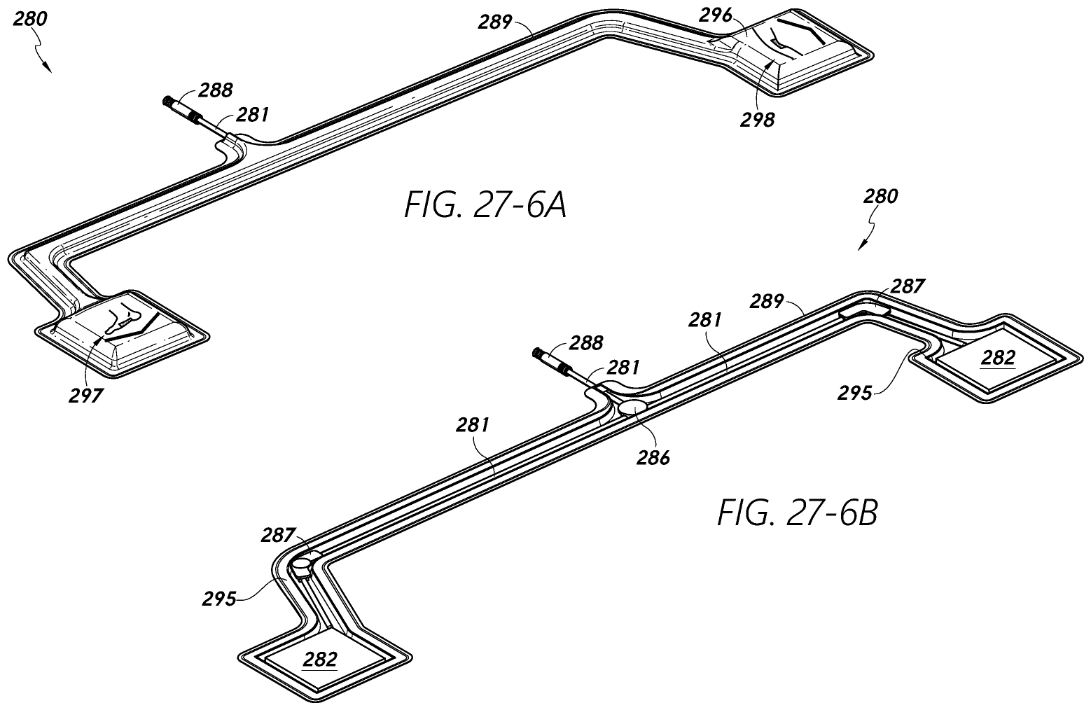

FIGS. 27-1, 27-2, 27-3, 27-4, and 27-5 show stages of a manufacturing process for manufacturing a heater assembly 280 in accordance with one or more embodiments. The referenced manufacturing process may involve, as shown in FIG. 27-1, separating/forming a sheet of vinyl or other polymer from a roll 291 or other sheet of such polymer material. For example, an area 292 of the polymer sheet/roll 291 may be cut out and/or stamped (e.g., steel ultrasonic horn-stamped) to form a bottom sheet 295 of the polymer, which will ultimately form the patient-facing or opposite-facing side/sheet of the polymer pouch of the heater assembly 280. In some embodiments, the polymer material/sheet 291 may comprise 0.005 inch vinyl, or other polymer sheet material.

The manufacturing process may further involve, as shown in FIG. 17-2, marking the sheet 295 with certain marks 293, 294 corresponding to the wire-management clips and heater pads of the heater assembly 280 for use in aligning such components of the heater assembly with the sheet 295. For example, in some implementations, the manufacturing process involves back-lighting the sheet 295 to identify the areas 293, 294, wherein the heater assembly 280, as shown in FIG. 27-3, is laid upon the sheet 295 in a manner as to align the respective components of the heater assembly 280 with the markings 293, 294 to facilitate proper placement/alignment of the heater assembly 280 with the sheet 295.

FIG. 27-4 shows the heater assembly 280, including the associated electronics thereof, placed on the sheet 295. In certain embodiments, adhesive may be implemented to secure the components of the heater assembly 280 in the illustrated position/placement. As shown in FIG. 27-5, the manufacturing procedure may further involve placing a top sheet 296 of the same polymer/plastic material as the bottom sheet 295, or alternatively a sheet of a different type of material, over the heater assembly 280 and bottom sheet 295.

FIGS. 27-6A and 27-6B show top and bottom perspective views, respectively, of the fully-assembled heater assembly 280 in accordance with one or more embodiments. For example, once the top sheet 296 has been placed over the bottom sheet 295, the sheets of polymer may be heat-sealed around the heater assembly 280 and/or cut to form a closed pouch (e.g., hermetically-sealed) around the heater assembly components, as described in detail herein. In some embodiments, the manufacturing process may further involve printing certain visual indicators/symbols 297, 298 on one or more sides of the pouch 289 to facilitate proper placement of the heater assembly 280 in the sleeve article/member to which the heater assembly 280 is intended to be coupled. For example, the heater assembly 280 may include a foot placement symbol 297, which may comprise an illustration/icon of a foot and/or a portion of a foot against which the foot heater pad is intended to be placed, thereby instructing the user as to which heating pad belongs in the area of the sleeve associated with the foot. Accordingly, a popliteal fossa image/icon 298 may likewise be printed/disposed on an area of the pouch 289 associated with the popliteal fossa heating pad to provide further guidance to the user.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others. “can.” “could.” “might.” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising.” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper” “lower.” “below,” “above,” “vertical.” “horizontal.” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less.” “more,” “greater,” and the like, are intended to encompass the concepts of equality. For example, “less” can mean not only “less” in the strictest mathematical sense, but also, “less than or equal to.”

What is claimed is:

1. A temperature-management device comprising:
a wearable sleeve including:
a foot portion configured to cover at least a portion of a sole of a foot of a patient;
a calf portion configured to cover at least a portion of a calf of the patient, the calf portion having one or more compression bladders associated therewith; and
a popliteal fossa portion configured to cover at least a portion of a popliteal fossa of the patient; and
a heater assembly configured to be secured to the wearable sleeve, the heater assembly including:
a foot heating pad;
a popliteal fossa heating pad; and
a physical connector coupling the foot heating pad and the popliteal fossa heating pad.

2. The device of claim 1, wherein the foot portion includes an inflatable heel pad configured to be fluidly coupled to at least one of the one or more compression bladders.

3. The device of claim 2, wherein the inflatable heel pad is fluidly coupled to a channel having a one-way valve associated therewith.

4. The device of claim 1, wherein the popliteal fossa portion of the wearable sleeve has a knee cut-out, such that the wearable sleeve is open around a knee of the patient.

5. The device of claim 1, wherein the physical connector of the heater assembly includes electrical wiring contained within a connector portion of a fluid-tight pouch.

6. The device of claim 1, wherein the calf portion of the wearable sleeve is detachable from one or more of the foot portion or the popliteal fossa portion.

7. The device of claim 1, wherein the foot heating pad has a visual indicator that corresponds with a corresponding indicator associated with the foot portion of the wearable sleeve.

8. The device of claim 1, wherein the wearable sleeve includes a closable cover configured to be covered over one or more portions of the heater assembly when the heater assembly is secured to the wearable sleeve.

9. The device of claim 1, further comprising an extension component configured to connect to the calf portion over a shin of the patient.

10. The device of claim 1, further comprising a fluid interface that is heat-sealed to the wearable sleeve and includes three fluidly-isolated conduits in fluid communication with three compression bladders of the one or more compression bladders, respectively.

11. The device of claim 1, wherein:
the heater assembly includes one or more color-coded indicators indicating a size of the heater assembly; and
the wearable sleeve includes one or more corresponding color-coded indicators on indicating a size of the wearable sleeve relative to the heater assembly.

12. The device of claim 1, wherein the wearable sleeve includes an adhesive strip configured to secure the heater assembly to the wearable sleeve.

13. The device of claim 1, wherein the wearable sleeve includes a heater assembly delineation pad on which the heater assembly is configured to be secured.

14. The device of claim 1, wherein the wearable sleeve and the heater assembly are configured such that, when the heater assembly is secured to the wearable sleeve:
the foot heating pad and the popliteal fossa heating pad are disposed on an inside of the wearable sleeve; and
at least a portion of the physical connector is disposed on an outside of the wearable sleeve.

15. The device of claim 14, wherein the at least a portion of the physical connector is configured to run outside of the calf portion of the wearable sleeve when the heater assembly is secured to the wearable sleeve.

16. A temperature-management device comprising:
a first heating pad;
a second heating pad;
electrical wiring electrically coupling the first heating pad and the second heating pad to an electrical connector; and
a pouch including:
a first heating pad portion covering at least a portion of the first heating pad;
a second heating pad portion covering at least a portion of the second heating pad; and
an elongate connector portion extending between the first heating pad portion and the second heating pad portion, the elongate connector portion covering at least a portion of the electrical wiring.

17. The device of claim 16, further comprising a means for indicating a usage of the temperature-management device, the means for indicating a usage comprising at least one of a mechanical indicator or one or more light sources.

18. The device of claim 16, further comprising control circuitry configured to:
determine a number of uses of the temperature-management device; and
activate an indicator in response to said determination.

19. The device of claim 16, further comprising one or more temperature sensors on a patient-facing side of one or more of the first heating pad and the second heating pad.

20. The device of claim 16, wherein the connector portion has one or more bends configured to allow for the connector portion to traverse a non-linear path.
